(19) United States
(12) Patent Application Publication
(10) Pub. No.: US 2025/0387242 A1
(43) Pub. Date: Dec. 25, 2025

(54) Method and Apparatus for a Prosthetic Device and a Prosthetic Coupling Device

(71) Applicant: Mark Alan Lewis, Grand Junction, CO (US)

(72) Inventor: Mark Alan Lewis, Grand Junction, CO (US)

(21) Appl. No.: 18/751,406

(22) Filed: Jun. 24, 2024

(51) Int. Cl. A61F 2/76; A61F 2/30

(52) U.S. Cl. CPC A61F 2/76; A61F 2002/30364

(57) ABSTRACT

A prosthetic coupling device includes an internal mount, a rotatable barrel formed around the internal mount, a locking ring formed between the internal mount and the rotatable barrel and mechanically affixed to the rotatable barrel, a horizontal ball bearing shaft formed through the internal mount housing a ball bearing set wherein the ball bearing set interfaces with the locking ring with the ball bearing set moving radially inward or outward based on the radial orientation of the locking ring and rotatable barrel, and a detachable coupler formed to fit within the mounting portion of the internal mount, the detachable coupler including a coupler detent formed on a radially external surface of the detachable coupler, wherein the ball bearing set interfaces with the coupler detent to lock the detachable coupler to the internal mount when the locking ring is radially oriented to move the ball bearing set radially inward.

### FIELD OF THE DISCLOSURE

The present disclosure generally relates to prosthetic devices. The present disclosure more specifically relates to a coupling device to operatively couple parts of a prosthetic device together via a twist action.

### BACKGROUND

Proper health care increases the life expectancy of patients. In some cases, patients are born with missing limbs or, due to an accident or a disease, lose a limb. The loss of a limb may require a prosthetic to replace the function of the missing limb so that the patient may realize a better quality of life and better health that is better than without the implementation of the prosthetic. In the case where the patient has lost a portion of the patient's lower extremities such as a lower portion of the patient's leg below the knee or even an upper portion of the leg above the knee, the prosthetic may be used to allow the patient to walk without the use of other mobility aides such as a wheelchair. However, these lower-extremity prosthetic devices must support the weight of the patient, be relatively easy to remove, and be comfortable for the patient.

### BRIEF DESCRIPTION OF THE DRAWINGS

It will be appreciated that for simplicity and clarity of illustration, elements illustrated in the Figures are not necessarily drawn to scale. For example, the dimensions of some elements may be exaggerated relative to other elements. Embodiments incorporating teachings of the present disclosure are shown and described with respect to the drawings herein, in which:

FIG. 1 is a graphic diagram front view of a prosthetic device with a prosthetic coupling device operatively coupling an upper pylon/proximate shaft portion and a lower pylon/distal shaft portion according to an embodiment of the present disclosure;

FIG. 2 is a graphic diagram front view of a prosthetic coupling device coupled to an upper pylon and a lower pylon according to another embodiment of the present disclosure;

FIG. 3 is a graphic diagram front view of a prosthetic coupling device according to another embodiment of the present disclosure;

FIG. 4 is a graphic diagram, side, cross-section view of the prosthetic coupling device according to another embodiment of the present disclosure;

FIG. 5 is a graphic diagram side view of a prosthetic coupling device without a detachable coupler of the prosthetic coupling device according to another embodiment of the present disclosure;

FIG. 6 is a graphic diagram, side, cross-section view of a rotatable barrel and an internal mount of the prosthetic coupling device without a detachable coupler of the prosthetic coupling device according to another embodiment of the present disclosure;

FIG. 7 is a graphic diagram front, perspective view of a locking ring of the prosthetic coupling device according to an embodiment of the present disclosure;

FIG. 8 is a graphic diagram top view of a locking ring of the prosthetic coupling device according to another embodiment of the present disclosure;

FIG. 9 is a graphic diagram, bottom, cross-section view of the rotatable barrel, locking ring, and an internal mount of the prosthetic coupling device according to an embodiment of the present disclosure;

FIG. 10 is a graphic diagram, bottom, cross-section view of the rotatable barrel, locking ring, and an internal mount of the prosthetic coupling device according to another embodiment of the present disclosure;

FIG. 11 is a graphic diagram, bottom, cross-section view of the rotatable barrel, and an internal mount of the prosthetic coupling device according to an embodiment of the present disclosure;

FIG. 12 is a graphic diagram, side, perspective view of an internal mount of the prosthetic coupling device according to an embodiment of the present disclosure;

FIG. 13 is a graphic diagram, side view of an internal mount of the prosthetic coupling device according to another embodiment of the present disclosure;

FIG. 14 is a graphic diagram side, cross-sectional view of an internal mount of the prosthetic coupling device according to another embodiment of the present disclosure;

FIG. 15 is a graphic diagram side, cross-section view of a detachable coupler of the prosthetic coupling device according to another embodiment of the present disclosure;

FIG. 16 is a graphic diagram side, perspective view of a lower spring cage of the prosthetic coupling device according to another embodiment of the present disclosure;

FIG. 17 is a graphic diagram top view of a lower spring cage of the prosthetic coupling device according to another embodiment of the present disclosure;

FIG. 18 is a front perspective view of a lower spring cage and an upper spring cage of the prosthetic coupling device according to an embodiment of the present disclosure;

FIG. 19 is a side, cross-section view of a prosthetic coupling device in an unlocked state according to another embodiment of the present disclosure;

FIG. 20 is a perspective view of a prosthetic coupling device according to another embodiment of the present disclosure;

FIG. 21 is a top view of the prosthetic coupling device 108 that includes a socket coupler according to another embodiment of the present disclosure;

FIG. 22 is a side view of a prosthetic coupling device showing the socket coupler operatively coupled to the internal mount of the prosthetic coupling device according to another embodiment of the present disclosure;

FIG. 23 is a side, cross-section view of a prosthetic coupling device according to another embodiment of the present disclosure;

FIG. 24 is a perspective view of a prosthetic coupling device according to another embodiment of the present disclosure;

FIG. 25 is a top view of a prosthetic coupling device according to another embodiment of the present disclosure;

FIG. 26 is a side, cross-section view of a prosthetic coupling device according to another embodiment of the present disclosure; and

FIG. 27 is a flow diagram of a method of manufacturing a prosthetic device according to an embodiment of the present disclosure.

The use of the same reference symbols in different drawings may indicate similar or identical items.

### DETAILED DESCRIPTION OF THE DRAWINGS

The following description in combination with the Figures is provided to assist in understanding the teachings disclosed herein. The description is focused on specific implementations and embodiments of the teachings and is provided to assist in describing the teachings. This focus should not be interpreted as a limitation on the scope or applicability of the teachings.

Prosthetic devices include a variety of artificial devices that are formed to replace a missing body part that was lost due to a disease, condition at birth, or accident. Prosthetic devices restore or provide mobility to those patients that have lost, for example, a limb. Leg prosthetics, for example, allow lower extremity amputee patients to walk instead of using other medical assisting devices such as a wheelchair. Mobility allows such an amputee to live a more self-reliant life that is more fulfilling than without the prosthetic device. Additionally, the patient wearing the prosthetic device is allowed to engage in activities that can provide for a healthier lifestyle thereby increasing the life expectancy of the patient.

Because prosthetic devices are connected to a patient's body during use, they may also be removed when not in use. This may require significant manipulation by the patient when putting on and taking off the prosthetic device. Still further, where the prosthetic device is a lower extremity prosthetic device, the patient may be required to put their weight on it thereby requiring that the prosthetic device not only be capable of carrying the patient's weight but also include connections that are secured and capable of being mechanically relied on by the patient. Additionally, the lower extremity prosthetic device may not be formed into a single piece and, instead, may include a plurality of pieces operatively coupled together to form the prosthetic device.

The present specification describes a prosthetic coupling device that includes an internal mount. A rotatable barrel is formed along the internal mount. In an embodiment, a locking ring is formed between the internal mount and the rotatable barrel and mechanically affixed to the rotatable barrel to be rotatably moveable with the rotatable barrel. In an embodiment, a horizontal ball bearing shaft is formed through the internal mount with the horizontal ball bearing shaft housing a ball bearing set. In an embodiment, the ball bearing set interfaces with the locking ring such that the ball bearing set is moved radially inward or outward based on the radial orientation of the locking ring and rotatable barrel. In an embodiment, a detachable coupler is formed to fit within the mounting portion of the internal mount. The detachable coupler may include, in an embodiment, a coupler detent formed on a radially external surface of the detachable coupler. In an embodiment, the ball bearing set may interface with the coupler detent to lock the detachable coupler to the internal mount when the locking ring is radially oriented to move the ball bearing set radially inward.

In an embodiment, the prosthetic device includes an upper spring cage and a lower spring cage with a spring placed between the upper spring cage and the lower spring cage. The spring cage spring being placed between the upper spring cage and the lower spring cage may, in an embodiment, create a biasing force against the rotatable barrel upwards to interface the locking ring with the ball bearing set to move the ball bearing set radially inward. In an embodiment, a locking ring spring may be placed between a lower surface of the locking ring and a surface formed on the internal mount. This locking ring spring may apply an upward biasing force against a bottom surface of the locking ring to force the locking ring upward, interfacing with the ball bearing set and forcing the ball bearing set radially inward to engage one or more detents on the detachable coupler thereby locking the detachable coupler to the remaining portions of the prosthetic coupling device.

In an embodiment, a bolt slot is formed on an exterior surface of the internal mount. Additionally, the prosthetic device includes a set screw having a head and a shaft, the shaft comprising outer threads, a bolt channel through the longitudinal axis of the shaft, and internal threads formed on an internal surface of the bolt channel. The set screw may interface with a bolt with the bolt placed coaxially within the bolt channel. In an embodiment, the length of the bolt extends past a terminal end of the set screw to extend into the bolt slot. In an embodiment, the bolt slot includes a horizontal portion, a first vertical slot formed at a first end of the horizontal portion of the bolt slot to receive an end of the bolt in a first radial orientation of the rotatable barrel, and a second vertical slot formed at a second end of the horizontal portion of the bolt slot to receive the end of the bolt in a second radial orientation of the rotatable barrel. In an embodiment, a bolt bearing may be formed on an end of the bolt to slide the bolt along the bolt channel.

In an embodiment, the prosthetic device may include an indicator ring formed onto an exterior surface of the internal mount. In an embodiment, the indicator ring is viewable to the patient when the ball bearing set is not interfacing with the coupler detents and the detachable coupler is not in a locked position with the internal mount and not viewable to the patient when the ball bearing set is interfacing with the coupler detents and the detachable coupler is in a locked position with the internal mount. This allows a patient to visually determine the locked and unlocked status of the prosthetic device.

During operation of the prosthetic coupling device the patient may rotate the rotatable barrel to disengage the internal mount from the detachable coupler. In an embodiment, the ball bearing set disengages with the coupler detents formed in the detachable coupler to unlock the detachable coupler from the internal mount when the locking ring is oriented to provide more space for the ball bearing set to be moved radially outward and away from the coupler detents.

In an embodiment, the prosthetic coupling device includes a registering slot to receive a registration pin formed on the detachable coupler to align the detachable coupler radially relative to the internal mount. This prevents the patient from coupling the distal portion of the prosthetic coupling device at an incorrect radial angle relative to the proximal portion of the prosthetic coupling device.

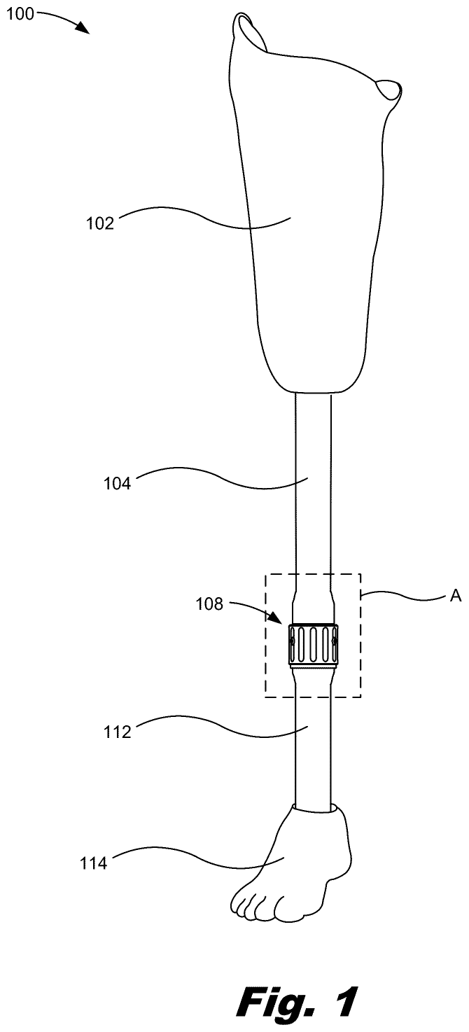

FIG. 1 is a graphic diagram front view of a prosthetic device 100 including a prosthetic coupling device 108 according to an embodiment of the present disclosure. FIG. 1 shows the prosthetic device 100 as a lower extremity prosthetic device that serves as a right leg or leg portion used by a patient to walk with. Additionally, the prosthetic device 100 is used by the patient to carry the patient's weight while the patient is standing, walking, and/or running. It is appreciated, however, that in other embodiments, the prosthetic coupling device 108 described herein may be used to operatively couple any number of portions or sections of any other type of prosthetic device together. Additionally, the prosthetic coupling device 108 may be used to couple any pylon (e.g., upper pylon/proximate shaft portion 104 and lower pylon/distal shaft portion 112) or two objects together and the present specification contemplates the use of the prosthetic coupling device 108 described herein for these other purposes. For ease of description and understanding, the prosthetic device 100 is described herein as a lower extremity or leg prosthetic device with the prosthetic coupling device 108 being used to operatively couple a first portion of the prosthetic device 100 to a second portion of the prosthetic device 100.

With reference to FIG. 1 and other figures in the present application, a distal end of the prosthetic device 100 is the end of the prosthetic device 100 furthest from the patient and includes the prosthetic foot 114. This prosthetic foot 114 may be made to look like a human foot and may be placed within a shoe or other footwear. Similarly, the use of the term “distal” when describing other elements and devices of the prosthetic device 100 including the prosthetic coupling device 108 is meant to be understood as a location on these elements and devices that are distant from the patient when the patient is wearing the prosthetic device 100. Similarly, the use of the term “proximate” when describing other elements and devices of the prosthetic device 100 including the prosthetic coupling device 108 is meant to be understood as a location on these elements and devices that are closest to the patient when the patient is wearing the prosthetic device 100. Additionally, the present specification may refer to a “patient” that may interact with the prosthetic device 100 and the prosthetic coupling device 108. However, the patient may refer to any user that uses or manipulates the prosthetic device 100 and/or prosthetic coupling device 108.

As shown in FIG. 1, the prosthetic device 100 includes a socket 102 portion. The socket 102 serves as an interface between a residual limb of the patient and the prosthetic device 100. In the example embodiment shown in FIG. 1, the socket 102 allows a patient to place his or her residual portion of a limb into the socket 102 which may be adjusted and customized for fit and comfort for the patient/amputee. The socket 102 may include other securing devices such as straps to operatively couple the socket 102 to the patient's residual limb. In an embodiment, a portion of the limb socket 102 into which the patient may place the patient's residual limb may be specifically fitted to that particular patient so that the limb socket 102 may conform to the surfaces of the patient's residual limb. Still further, in some embodiments, the socket 102 may include a vacuum pump that creates a vacuum pressure between the socket 102 and the patient's residual limb.

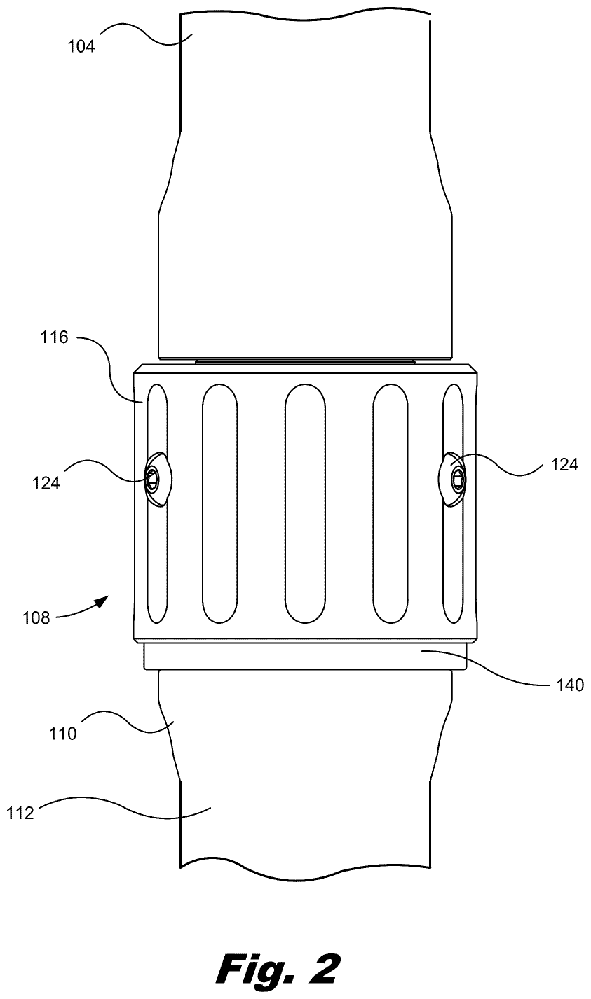

The socket 102, in an embodiment, may be operatively coupled to an upper pylon/proximate shaft portion 104 of the prosthetic device 100. In an embodiment, the upper pylon/proximate shaft portion 104 may be made of a durable material such as metal that can bear the weight of the patient. Such materials may include titanium, aluminum, copper, steel, magnesium, alloys of these metals, along with other metals. In an embodiment, the upper pylon/proximate shaft portion 104 may be made of carbon-fiber weave or carbon-fiber-reinforced polymer. The present specification further contemplates that wall thicknesses of the lower pylon/distal shaft portion 112, the upper pylon/proximate shaft portion 104, the internal mount described herein (e.g., FIG. 2, 132 for example), and the detachable coupler (e.g., FIG. 2, 140 for example) may be selected to provide a sufficient structural integrity to withstand the weight from the patient and the structural strains placed on the prosthetic device 100 and the prosthetic coupling device 108.

In an embodiment, the length of the upper pylon/proximate shaft portion 104 of the prosthetic device 100 may be selected based on the patient's ability to access the prosthetic coupling device 108 shown in FIG. 1 such that the patient can reach and access the prosthetic coupling device 108 described herein in order to manipulate the prosthetic coupling device 108 as described herein. In an embodiment, the length of the upper pylon/proximate shaft portion 104 combined with the length of a lower pylon 112, a height of the prosthetic foot 114, and a length of the prosthetic coupling device 108 may also be selected based on the length of the patient's other length so that the total length of the prosthetic device 100 may match the length of the patient's other leg. It is appreciated that, where the patient is a double amputee in their lower extremities, the length of the prosthetic device 100 described herein may match the length of the other prosthetic device 100. In an embodiment, the length of the upper pylon/proximate shaft portion 104 as well as the length of the lower pylon/distal shaft portion 112 and height of the prosthetic foot 114 may be determined and selected by a doctor or other prosthetic building professional to facilitate a maximum comfort for the patient.

As described herein, the prosthetic device 100 includes the prosthetic coupling device 108 described herein. The prosthetic coupling device 108 couples a first portion of the prosthetic device 100 to a second portion of the prosthetic device 100. In the example embodiment, shown in FIG. 1, the prosthetic coupling device 108 operatively couples the upper pylon/proximate shaft portion 104 to the lower pylon 112 with its prosthetic foot 114. It is appreciated, as well, that the prosthetic coupling device 108 does not need to be operatively coupled to an upper pylon/proximate shaft portion 104 and a lower pylon/distal shaft portion 112 and can, instead be coupled directly to a proximate or distal end of a prosthetic limb. In an example embodiment, various attachment scenarios allow for the prosthetic coupling device 100 to be directly coupled to the socket 102 or the prosthetic foot 114 and the present specification contemplates these other attachment scenarios.

As described herein, the prosthetic coupling device 108 is configured to secure the upper pylon/proximate shaft portion 104 to the lower pylon/distal shaft portion 112 via a rotation of a rotatable barrel of the prosthetic coupling device 108. This relatively quick action of rotating the rotatable barrel allows a patient to quickly uncouple and couple the lower pylon/distal shaft portion 112 to the upper pylon/proximate shaft portion 104. In an embodiment, multiple lower pylon/distal shaft portion 112 and prosthetic foot 114 combinations may be purchased and used by the patient. In an example embodiment, the patient may own a first lower pylon/distal shaft portion 112 and prosthetic foot 114 that has a first type of shoe on the prosthetic foot 114 such as a tennis shoe or sneaker. In this example embodiment, the patient may also own a second lower pylon/distal shaft portion 112 and prosthetic foot 114 combination that includes a dress shoe on the prosthetic foot 114. Still further, the patient, in an example embodiment, may also own a third lower pylon/distal shaft portion 112 that includes a running blade or other running prosthetic coupled to a distal end of the lower pylon/distal shaft portion 112 or distal end of the prosthetic coupling device 108. With the prosthetic coupling device 108, the patient may easily switch from the first lower pylon/distal shaft portion 112 and prosthetic foot 114/shoe combination with the second lower pylon/distal shaft portion 112 and prosthetic foot 114/shoe or the third lower pylon/distal shaft portion 112 and running blade combination. This is done by the patient rotating the rotatable barrel of the prosthetic coupling device 108 in a first direction, removing the first lower pylon/distal shaft portion 112 and prosthetic foot 114/shoe combination from the prosthetic coupling device 108 and inserting the second lower pylon/distal shaft portion 112 and prosthetic foot 114/shoe combination or the third lower pylon/distal shaft portion 112 and running blade combination into the prosthetic coupling device 108. The patient may then turn the rotatable barrel in a second direction to lock the other (e.g., second or third) lower pylon 108 and prosthetic foot 114/shoe or running blade combination to the prosthetic coupling device 108 and the upper portions of the prosthetic device 100. It is also clear that other, differently fitted, lower pylon/distal shaft portion 112 and prosthetic foot 114 combinations may be owned by the patient to quickly switch out different footwear when needed for example. Additionally, the prosthetic coupling device 108 allows the patient to know when the lower pylon/distal shaft portion 112/prosthetic foot 114 is securely locked to the prosthetic device 100. For example, visual indicators present on the prosthetic coupling device 108 may visually inform the patient of the locked or unlocked state of the prosthetic coupling device 108 thereby preventing the patient from accidentally using (e.g., walking or running) the prosthetic device 100 in an unlocked state.

The lower pylon/distal shaft portion 112 is used to operatively couple the prosthetic foot 114 to the prosthetic coupling device 108 and also to the upper pylon/proximate shaft portion 104 and socket 102. In an embodiment, the lower pylon/distal shaft portion 112 may be made of a durable material such as metal that can bear the weight of the patient. Such materials may include titanium, aluminum, copper, steel, magnesium, alloys of these metals, along with other metals. In an embodiment, the upper pylon/proximate shaft portion 104 may be made of carbon-fiber weave or carbon-fiber-reinforced polymer. The lower pylon/distal shaft portion 112 may be operatively coupled to a prosthetic foot 114 or other distal part of the prosthetic device 100 such as a running blade using other types of mechanical coupling devices.

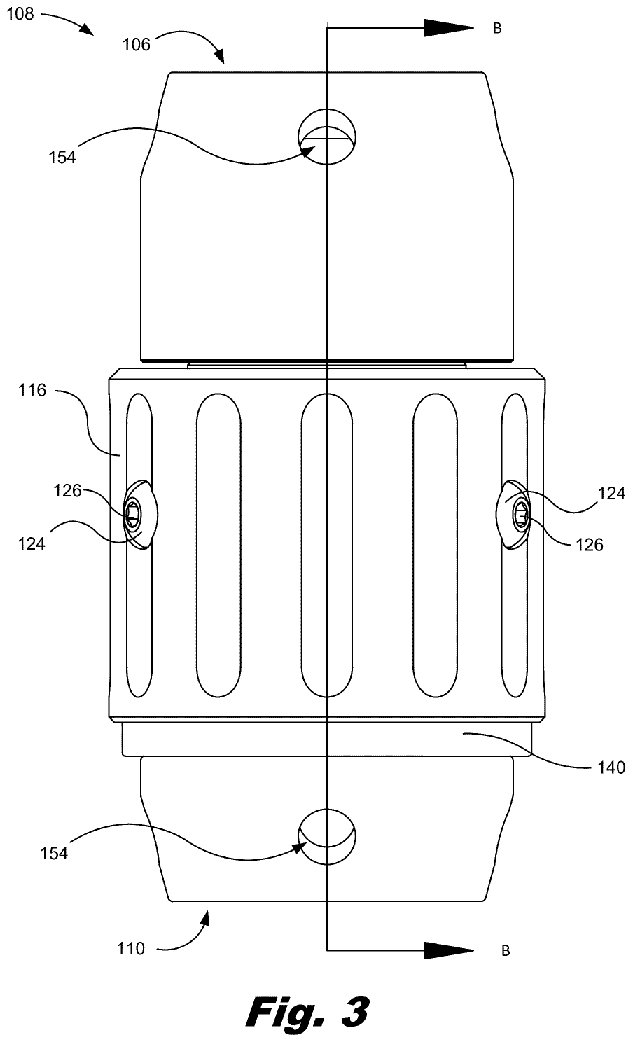

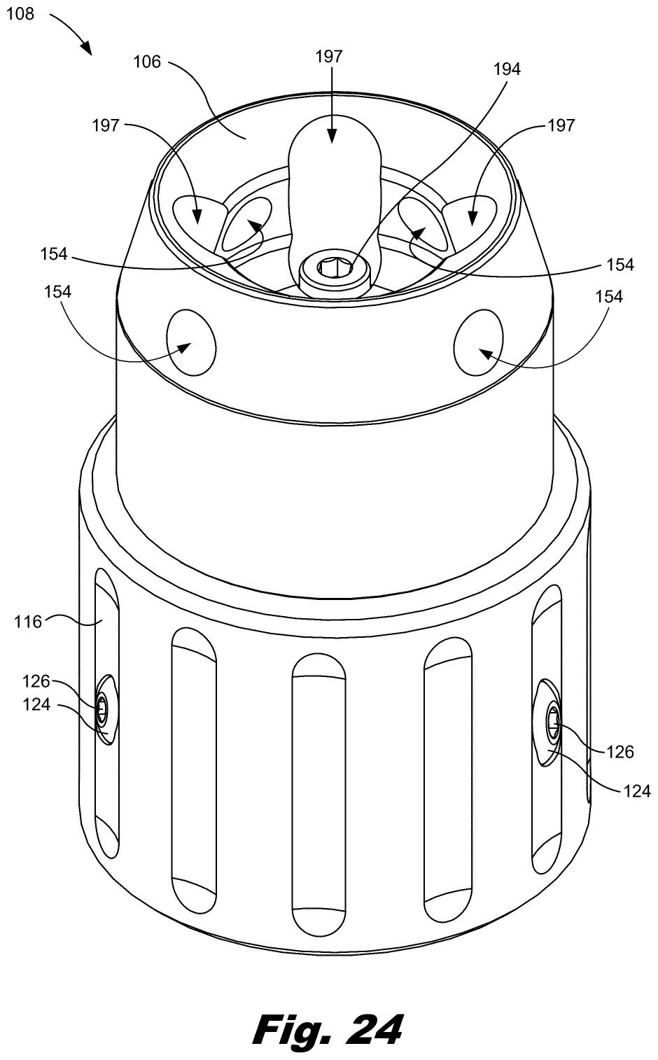

FIG. 1 also includes a section window “A” indicating a section of the prosthetic device 100 including the prosthetic coupling device 108 shown in FIG. 2. FIG. 2 is a graphic diagram front view of a prosthetic coupling device 108 coupling an upper pylon/proximate shaft portion 104 and a lower pylon/distal shaft portion 112 together according to another embodiment of the present disclosure. FIG. 2 shows that the upper pylon/proximate shaft portion 104 is operatively coupled to an internal mount (not shown) of the prosthetic coupling device 108. The upper pylon/proximate shaft portion 104 may, therefor, in an embodiment, form a monolithic part with the internal mount described herein. FIG. 2 also shows that a lower pylon/distal shaft portion 112 is operatively coupled to a detachable coupler 140 of the prosthetic coupling device 108. In an embodiment, the lower pylon/distal shaft portion 112 forms a monolithic part with detachable coupler 140 as described herein. In an embodiment, the prosthetic coupling device 108 includes an upper socket coupler (e.g., 106, FIG. 3) such as a pyramid coupler shown in FIG. 24, for example. In an embodiment, the prosthetic coupling device 108 includes a lower socket coupler (e.g., 110, FIG. 3) similar to the coupler described herein in connection with FIG. 24, for example.

During operation, the patient may couple the lower pylon/distal shaft portion 112 and the detachable coupler 140 to the remaining portions of the prosthetic coupling device 108 including the upper pylon/proximate shaft portion 104 by inserting the detachable coupler 140 into the bottom side of the prosthetic coupling device 108. The patient may then rotate the rotatable barrel 116 in a first direction (e.g., clockwise from the perspective of the patient above the rotatable barrel 116 in an embodiment) to lock the detachable coupler to the internal mount within the prosthetic coupling device 108. To remove the lower pylon 108 and the detachable coupler 140 from the internal mount, the patient may rotate the rotatable barrel 116 in a second direction (e.g., counterclockwise from the perspective of the patient above the rotatable barrel 116). As described herein, this connection of the lower pylon/distal shaft portion 112 to and decoupling of the lower pylon/distal shaft portion 112 from the prosthetic coupling device 108 allows the patient to easily remove this portion of the prosthetic device 100 and, in an embodiment, replace it with a different lower portion (e.g., the lower pylon/distal shaft portion 112 and prosthetic foot 114/running blade) of the prosthetic device 100 as described herein. It is appreciated, however, that this prosthetic device 100 may be used for different prosthetic devices and the present specification contemplates the use of these other uses of the prosthetic coupling device 108 described herein.

FIG. 2 also shows a number of set screws 124 formed through the rotatable barrel 116 of the prosthetic coupling device 108. As described in more detail herein, each set screw 124 may be used to prevent over rotation of the rotatable barrel 116 when the patient is locking and unlocking the detachable coupler to and from the remaining portions of the prosthetic coupling device 108. In an example embodiment, these set screws 124 may set a rotational range of the rotatable barrel 116 to, for example, a quarter turn in order for the patient to couple and decouple the detachable coupler to and from the remaining portions of the prosthetic coupling device 108. In an embodiment, the set screws 124 are tapped into the rotatable barrel 166 at a generally similar horizontal plane.

FIG. 3 is a graphic diagram front view of a prosthetic coupling device 108 according to another embodiment of the present disclosure. FIG. 3, unlike FIG. 2, shows the prosthetic coupling device 108 being uncoupled from the upper pylon/proximate shaft portion 104 at the proximal end of the prosthetic coupling device 108 and uncoupled from the lower pylon/distal shaft portion 112 at a distal end of the prosthetic coupling device 108. In an embodiment, the upper socket coupler 106 of the prosthetic coupling device 108 is used to operatively couple the upper pylon/proximate shaft portion 104 to the prosthetic coupling device 108. Similarly, the prosthetic coupling device 108 includes a lower socket coupler 110 operatively coupled to the detachable coupler 140 used to operatively couple the lower pylon/distal shaft portion 112 to a distal end of the prosthetic coupling device 108.

In an embodiment, the upper socket coupler 106 and the lower socket coupler 110 may each include socket coupler coupling holes 154. Each of the socket coupler coupling holes 154 may be used to receive a coupling device (not shown) such as a bolt, screw, set screw, or other coupling device in order to secure the upper pylon/proximate shaft portion 104 to the proximal end of the prosthetic coupling device 108 and the lower pylon/distal shaft portion 112 to a distal end of the prosthetic coupling device 108. It is appreciated that any type of coupling device may be used to secure the upper pylon/proximate shaft portion 104 to the proximal end of the prosthetic coupling device 108 and the lower pylon/distal shaft portion 112 to the distal end of the prosthetic coupling device 108 and the present specification contemplates these other coupling devices. In an embodiment, the coupling device used to secure the lower pylon/distal shaft portion 112 and upper pylon/proximate shaft portion 104 to the prosthetic coupling device 108 may be secured into the socket coupler coupling holes 154 such that a patient may not be capable of removing the coupling devices. This may prevent the dismantling of the upper pylon/proximate shaft portion 104 and lower pylon/distal shaft portion 112 from the prosthetic coupling device 108 during use by the patient. In an embodiment, the coupling devices may only be removed using a proprietary tool that interfaces with the coupling devices such that a patient would be incapable of manipulating these coupling devices thereby preventing the removal of the upper pylon/proximate shaft portion 104 and lower pylon/distal shaft portion 112 from the prosthetic coupling device 108. Thus, in an embodiment, the coupling devices may include a screw drive or head that requires a specific wrench usually not owned or used by the patient such that the patient is prevented from adjusting the fastening devices. This prevents a patient from altering the prosthetic device apart from the prosthetic coupling device 108 without supervision from a trained orthotic or other prosthetic fitting personnel.

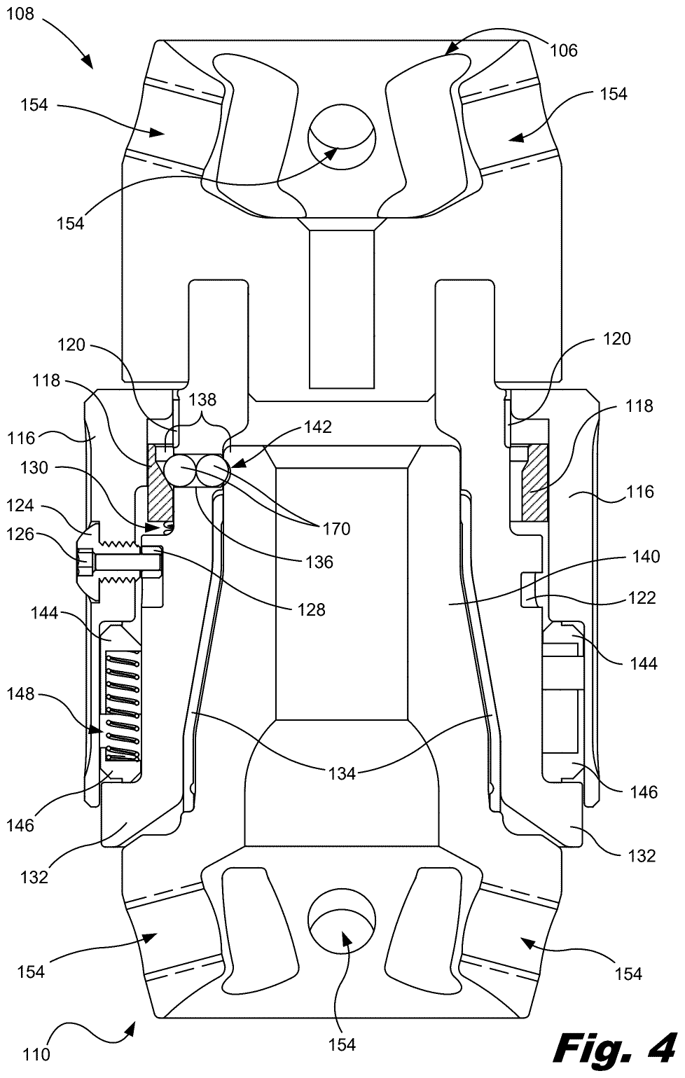

FIG. 3 also includes a section line “B” indicating a cross-sectional plane of the prosthetic coupling device 108 as shown in FIG. 4. FIG. 4 is a graphic diagram, side, cross-section view of the prosthetic coupling device 108 according to another embodiment of the present disclosure. FIG. 4 shows the rotatable barrel 116 fully rotated in a clockwise direction, for example, such that the detachable coupler 140 is coupled to the prosthetic coupling device 108. This orientation in FIG. 4 of the prosthetic coupling device 108 shows the prosthetic coupling device 108 in a locked state or position with the detachable coupler 140 locked into the internal mount 132 as described herein.

The prosthetic coupling device 108 may include the detachable coupler 140 operatively couplable to the prosthetic coupling device 108 by insertion of the detachable coupler 140 into an internal space formed within the internal mount 132 as shown in FIG. 4. As described herein, the detachable coupler 140 includes a lower socket coupler 110 that is used to operatively couple the lower pylon/distal shaft portion (e.g., FIG. 2, 112) to the prosthetic coupling device 108 via the socket coupler coupling holes 154 described herein. Additionally, the prosthetic coupling device 108 includes an upper socket coupler 106 that is used to operatively couple the upper pylon/proximate shaft portion (e.g., FIG. 3, 104) to the prosthetic coupling device 108 via the socket coupler coupling holes 154 described herein. In an embodiment, the upper socket coupler 106 may form a monolithic piece with the internal mount 132. In another embodiment, the upper socket coupler 106 may be coupled to the internal mount 132 using a glue or a fastening device such as a screw or bolt. In yet another embodiment, the upper socket coupler 106 may be operatively coupled to the internal mount 132 via a friction welding process.

In an embodiment, the detachable coupler 140 may be formed to fit within an internal mount 132 of the prosthetic coupling device 108 such that the detachable coupler 140 and internal surface of the internal mount 132 have an engineering fit that allows for their dismantling and coupling by a patient. For example, the engineering fit between the outer surface of the detachable coupler 140 and the internal surface of the internal mount 132 may be a close running clearance fit, a sliding clearance fit, or a location clearance fit. It is appreciated that any type of clearance fit or any other engineering fit may be formed between the outer surface of the detachable coupler 140 and the internal surface of the internal mount 132 and the present specification contemplates these types of other fits. It is appreciated that because the interface between the outer surface of the detachable coupler 140 and the internal surface of the internal mount 132 are to bear the patient's weight within the prosthetic coupling device 108, the selected engineering fit may prevent movement of the detachable coupler 140 within the internal mount 132 when coupled together so that the detachable coupler 140 doesn't move when the patient is using the prosthetic coupling device 108 to stand, walk, or run with.

In an embodiment, the detachable coupler 140 may include an outer plastic sleeve 134 formed on an outer surface of the detachable coupler 140. In an embodiment, the outer plastic sleeve 134 may be made of an acetal plastic or other similar material that has a low surface energy that has a relatively high wear resistance and high strength. Additionally, a low surface energy of the outer plastic sleeve 134 allows the detachable coupler to be easily slid into and out of the internal mount 132 of the prosthetic coupling device 108 during operation by the patient. In an embodiment, this outer plastic sleeve 134 may be milled to fit around the outer surface of the detachable coupler 140. In an embodiment, the outer plastic sleeve 134 may be injection molded onto the detachable coupler 140. In another embodiment, the outer plastic sleeve 134 may be coupled to the outer surface of the detachable coupler 140 via a glue or other adhesive. In an embodiment, the glue or adhesive may include RelTek® Bondit® B-4682TH or other similar adhesives that adhere the outer plastic sleeve 134 to the metal (e.g. aluminum such as 2024-T351 aluminum and similar alloys) of the detachable coupler 140. The high strength and wear resistance of the outer plastic sleeve 134 allows for the multiple coupling and uncoupling of the detachable coupler 140 from the internal mount 132 of the prosthetic coupling device 108 with reduced amounts of wear and tear between the detachable coupler 140 and the internal mount 132.

The inclusion of the outer plastic sleeve 134 acts as a lubricant, facilitating easy separation of detachable coupler 140 from the internal mount 132 of the prosthetic coupling device 108 for easier use by the patient. Additionally, it is appreciated that other types of plastics or materials different from the acetal plastic of the outer plastic sleeve 134 may be used and the present specification contemplates the use of these other materials. In an embodiment, for example, a brass sleeve 134 could also provide similar properties as the acetal plastic of the outer plastic sleeve 134. It is appreciated that surface treatments other than the outer plastic sleeve 134 may be placed over the internal mount 132 to provide a low friction surface between the internal mount 132 of the detachable coupler 140 as well as any other internal surfaces within the internal mount 132.

In embodiments herein, the prosthetic coupling device 108 includes a locking ring 118 used to push one or more ball bearings 170 of a ball bearing set 138 into one or more coupler detents 142 formed onto an outer surface of the detachable coupler 140. In an embodiment, the locking ring 118 is made of a plastic such as an acetal plastic. In an embodiment, the locking ring 118 may be made of a low friction material that allows the ball bearings 170 of the ball bearing set 138 to pass easily over the surface of the locking ring 118 during operation of the prosthetic coupling device 108 as described herein. In an embodiment, the locking ring 118 includes one or more sloped surfaces that, as the locking ring 118 is pushed upwards and is at a specific degree of rotation about the internal mount 132, a thicker portion of the locking ring 118 forces a series of ball bearings 170 of one or more ball bearing sets 138 through their respective horizontal ball bearing shafts 136. As the ball bearings 170 are forced through the horizontal ball bearing shafts 136, a terminal ball bearing of the ball bearing set 138 may extend past an inner surface of the internal mount 132 and interface with the one or more coupler detents 142 formed on the outer surface of the detachable coupler 140. In the embodiment shown in FIG. 4, the coupler detents 142 are formed on a proximal end of the detachable coupler 140 with the horizontal ball bearing shafts 136 and ball bearing set 138 being aligned with the coupler detents 142 when the detachable coupler 140 is fully seated within the internal mount 132.

As shown in FIG. 4, the locking ring is formed around the internal mount 132 and within the rotatable barrel 116. In an embodiment, the locking ring 118 is operatively coupled to the rotatable barrel 116 such that rotation of the rotatable barrel 116 causes the locking ring 118 to also rotate the same degree. In an embodiment, the locking ring 118 may be glued to an inner surface of the rotatable barrel 116. In an embodiment, the locking ring 118 may be secured to the inner surface of the rotatable barrel 116 via one or more coupling devices such as screws, bolts, and the like. In an embodiment, the locking ring 118 may be coupled to the inner surface of the rotatable barrel 116 via an engineering fit that prevents independent rotation of the locking ring 118 relative to the rotatable barrel 116 and/or prevents the locking ring 118 from moving vertically independent from the rotatable barrel 116. In an embodiment, this engineering fit may include an interference fit such as a press fit, a driving fit, or a forced fit. It is appreciated that any type of engineering fit between the locking ring 118 and the rotatable barrel 116 may be used in order to prevent independent rotation of the locking ring 118 relative to the rotatable barrel 116 during operation by the patient. In an alternative embodiment, the locking ring 118 may have one or more flat surfaces formed on an external surface of the locking ring 118 that interface, mechanically, with matching flat surfaces formed on the internal surface of the rotatable barrel 116. In this embodiment, as the rotatable barrel 116 is rotated, the flat surfaces formed on the internal surface of the rotatable barrel 116 mechanically interface with the flat surfaces formed on the external surface of the locking ring 118 such that the locking ring 118 rotates with the rotation of the rotatable barrel 116.

The locking ring 118 is formed such that as the locking ring 118 rotates about the internal mount 132, the ball bearing set 138 is allowed to move radially outward through the horizontal ball bearing shafts 136 such that they disengage with the coupler detents 142 formed on the outer surface of the detachable coupler 140. In an embodiment, a width of the locking ring 118 is reduced at certain locations along the circumference of the locking ring 118 such that extra space is provided for the movement of the ball bearings 170 of the ball bearing set 138 to move into and, therefore, move radially outward within the horizontal ball bearing shafts 136. In an embodiment, the movement of the ball bearing sets 138 radially outward through the horizontal ball bearing shafts 136 does not occur until the rotation of the locking ring 118 aligns the locations along the circumference where the width of the locking ring 118 is reduced. This coincides with the bolt 126 and set screw 124 assembly reaching a terminal end of the bolt slot 122. In an embodiment, this may be where the second end of the horizontal portion of the bolt slot 122 is reached by the set screw 124 and where the set screw 124 is movable into the second vertical slot formed at a second end of the horizontal portion of the bolt slot 122 as described herein.

In an embodiment, the locking ring 118 interfaces with a locking ring spring 130 at a bottom surface of the locking ring 118. The locking ring spring 130 may be biased to force the locking ring 118 upwards and towards an interior surface of the rotatable barrel 116. As described herein, under certain orientations of the locking ring 118 and rotatable barrel 116, as the locking ring 118 is forced upwards by the locking ring spring 130, the ball bearings 170 of the ball bearing set 138 are shunted radially into the horizontal ball bearing shafts 136 causing the ball bearings 170 to be forced into the coupler detents 142 to lock the detachable coupler 140 into the internal mount 132 as described herein.

In an embodiment, as the rotatable barrel 116 is pressed down and rotated (e.g., rotated counterclockwise) a distance by the patient, the set screw 124 travels within a bolt slot 122. As the patient releases pressure applied to the rotatable barrel 116, the rotatable barrel 116 may be pushed upwards by action of, at least, the locking ring spring 130. In an embodiment, the locking ring 118, when rotated, has cavities or surfaces that allow the ball bearings 170 to fall or pass into, thereby allowing the ball bearings 170 of the ball bearing set 138 to move radially outwards. This disengages the ball bearing sets 138 from their respective coupler detents 142 formed in the outer surface of the detachable coupler 140, and the detachable coupler 140 may be removed from the remaining portions of the prosthetic coupling device 108.

Where the detachable coupler 140 is removed from the remaining portion of the prosthetic coupling device 108, the lower pylon/distal shaft portion 112 and prosthetic foot 114 operatively coupled to the detachable coupler 140 is also removed thereby allowing the patient to switch out the detachable coupler 140/lower pylon/distal shaft portion 112/prosthetic foot 114 assembly with another detachable coupler 140/lower pylon/distal shaft portion 112/prosthetic foot 114 assembly where needed by the patient.

As described herein, the internal mount 132 includes a bolt slot 122 formed along an outer surface of the internal mount 132. In an embodiment, the bolt slot 122 may be formed, generally, in a horizontal direction with a first vertical slot extending above the bolt slot 122 at a first end and a second vertical slot extending above the bolt slot 122 at a second end of the bolt slot 122. The bolt slot 122 may interface with the set screw 124/bolt 126 assembly which is passed through the rotatable barrel 116. By setting the length of the horizontal portion of the bolt slot 122, the degree of rotation of the rotatable barrel 116 is also set. Therefore, where the horizontal portion of the bolt slot 122 extends a quarter portion around the internal mount 132, the rotatable barrel 116 is allowed to only be rotated a quarter turn. It is appreciated that the length of the horizontal portion of the bolt slot 122 may be increased or decreased in order to facilitate the operation of the prosthetic coupling device 108 as described herein and further sets the degree of rotation of the rotatable barrel 116 by the patient to lock or unlock the detachable coupler 140 to the prosthetic coupling device 108 as described herein.

In an embodiment, the prosthetic coupling device 108 further includes an upper spring cage 144 and a lower spring cage 146 with one or more spring cage springs 148 formed between the upper spring cage 144 and lower spring cage 146. In an embodiment, each of the upper spring cage 144 and lower spring cage 146 may be made of a plastic such as an acetal plastic. In an embodiment, the upper spring cage 144 and lower spring cage 146 may be made of low friction material that allows moving elements of the prosthetic coupling device 108 such as the rotatable barrel 116 to move easily over the surfaces of the upper spring cage 144 and lower spring cage 146. In an embodiment, the upper spring cage 144 and lower spring cage 146 may be mechanically affixed to an outer surface of the internal mount 132 such that the upper spring cage 144 and lower spring cage 146 do not rotate with the rotation of the rotatable barrel 116, for example. In these embodiments, the upper spring cage 144 and lower spring cage 146 may be operatively coupled to the external surface of the internal mount 132 using a coupling device (bolt, screw, nail, etc.), a glue, or via an engineering fit that prevents movement of the upper spring cage 144 and lower spring cage 146 relative to the internal mount 132.

The upper spring cage 144 and lower spring cage 146 may be, generally, in the form of a ring that each are oriented around an outer surface of the internal mount 132. In an embodiment, an upper surface of the upper spring cage 144 may be forced against a lower surface formed on an internal surface of the rotatable barrel 116 using the one or more spring cage springs 148. Additionally, a lower surface of the lower spring cage 146 may be forced against a shelf or other surface formed on the external surface of the internal mount 132 using the one or more spring cage springs 148. Consequently, the upper spring cage 144, lower spring cage 146, and one or more spring cage springs 148 are used to apply a biased force against the rotatable barrel 116 such that the rotatable barrel 116 is forced upward away from the internal mount 132. This upward biasing force against the rotatable barrel 116 by the use of the upper spring cage 144, lower spring cage 146, and one or more spring cage springs 148 is in addition to the force applied to the rotatable barrel 116 by the locking ring spring 130 that also forces the locking ring 118 upwards and against the internal surface of the rotatable barrel 116 as described herein.

As shown in FIG. 4, the set screw 124 with its bolt 126 are passed through the rotatable barrel 116 and engages with the first vertical slot formed at a first end of the horizontal portion of the bolt slot 122. In this orientation, the prosthetic coupling device 108 is in a locked state with the detachable coupler 140 being locked into the internal mount 132 of the prosthetic coupling device 108. During operation, the patient may press down on the rotatable barrel 116 to pass the bolt 126 and set screw 124 downward along the vertical slot and into the horizontal portion of the bolt slot 122. By pressing down on the rotatable barrel 116, the patient overcomes the spring bias from the one or more locking ring springs 130 and one or more spring cage springs 148. As the patient rotates the rotatable barrel 116 in a direction (e.g., in a counterclockwise direction), the bolt 126 and a bolt bearing 128 formed on the end of the bolt 126 are passed along the entire length of the horizontal portion of the bolt slot 122 until this assembly reaches the second end of the horizontal portion of the bolt slot 122 and to a second vertical slot formed at a second end of the horizontal portion of the bolt slot 122. In an embodiment, this second vertical slot formed at the second end of the horizontal portion of the bolt slot 122 may have a vertically lower terminal end than the first vertical slot preventing the rotatable barrel 116 from returning to a maximum height as shown in FIG. 4 as the prosthetic coupling device is in the locked state. When the set screw 124/bolt 126 assembly is placed within the second vertical slot of the bolt slot 122, the prosthetic coupling device 108 is in an unlocked state.

In an embodiment, the set screw 124 may have a channel formed through the entire axial length of the set screw 124. This channel may include threading to receive the bolt 126 along the axial length of the set screw 124 such that the bolt 126 is formed coaxially with the set screw 124. In an embodiment, the length of the bolt 126 exceeds the length of the set screw 124 such that the bolt 126 extends past the length of the set screw 124 a distance. As shown in FIG. 4, the bolt 126 extends past the set screw 124 and past an internal surface of the rotatable barrel 116 thereby being the only part of the set screw 124/bolt 126 assembly that enters into the bolt slot 122 formed on the external surface of the internal mount 132. Additionally, in an embodiment, the bolt 126 may be fitted with a bolt bearing 128. The bolt bearing 128 may be allowed to rotate about the axis of the bolt 126 such that as the rotatable barrel 116 is rotated, the bolt bearing 128 engages with the surfaces of the bolt slot 122 to allow for the bolt 126 and bolt bearing 128 to pass along the surface of the bolt slot 122 to reduce friction between the bolt 126 and the surfaces of the bolt slot 122. Additionally, the diameter of the bolt bearing 128 may be selected such that as the bolt 126 and bolt bearing 128 reach the vertical portions of the bolt slot 122, the bolt bearing 128 fits and is seated within these vertical portions of the bolt slot 122.

In an embodiment, the prosthetic coupling device 108 may include an indicator ring 120. In an embodiment, the indicator ring 120 may be used by the patient to determine the locked or unlocked state of the prosthetic coupling device 108. As described herein, when the prosthetic coupling device 108 is in a locked state as shown in FIG. 4, the rotatable barrel 116 is in a maximum vertical position. With the rotatable barrel 116 in this maximum vertical position, the indicator ring 120 is not visible to the patient because the rotatable barrel 116 is physically covering the indicator ring 120. However, as described herein, when the prosthetic coupling device 108 is in an unlocked state, the position of the rotatable barrel 116 is altered such that the rotatable barrel 116 is at a lower position than when the prosthetic coupling device 108 is in a locked state. With the rotatable barrel 116 at a lower position, the indicator ring 120 is made visible by the patient. Therefore, in an embodiment, where the patient can see the indicator ring 120, the patient will understand that the prosthetic coupling device 108 is in an unlocked state and that the detachable coupler 140/lower pylon/distal shaft portion 112/prosthetic foot 114 assembly may be removed from the remaining portions of the prosthetic coupling device 108. Even further, where the patient sees the indicator ring 120, the patient may understand that the patient should not place weight on the prosthetic device (e.g., FIG. 1, 100) or otherwise walk using the prosthetic device due to the detachable coupler 140 not being operatively locked to the remaining portions of the prosthetic coupling device 108 as described herein.

In an embodiment, the indicator ring 120 may be glued to the external surface of the internal mount 132. In another embodiment, the indicator ring 120 may be force fit onto the external surface of the internal mount 132 using an engineering fit that prevents the removal of the indicator ring 120 from the internal mount 132. In another embodiment, the indicator ring 120 may be operatively coupled to the external surface of the internal mount 132 using any type of fastening device such as a nail, screw, or bolt among others.

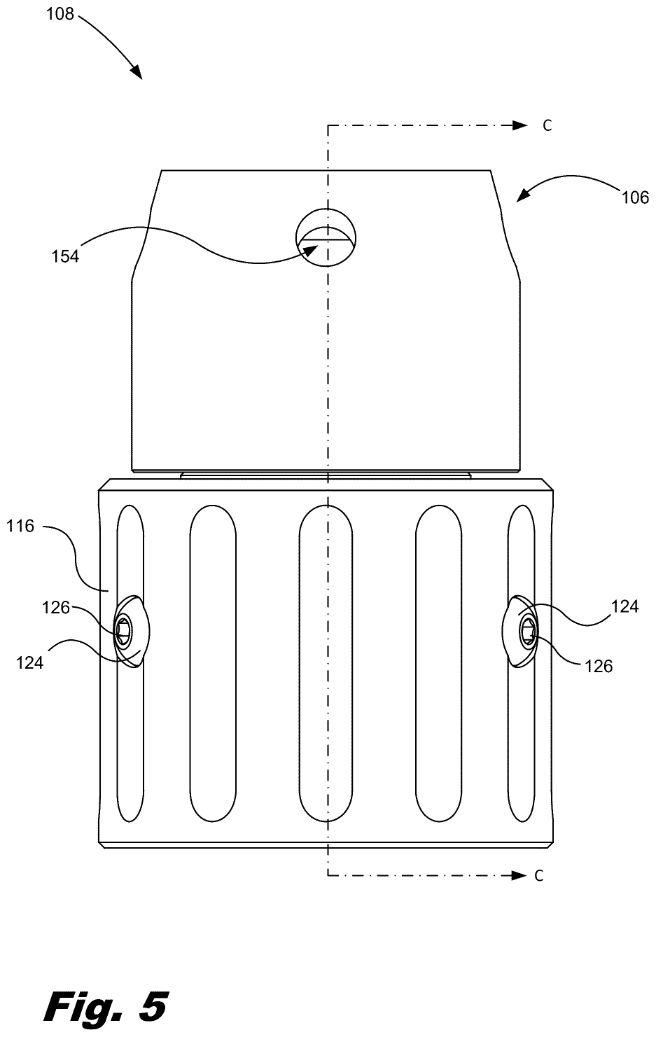

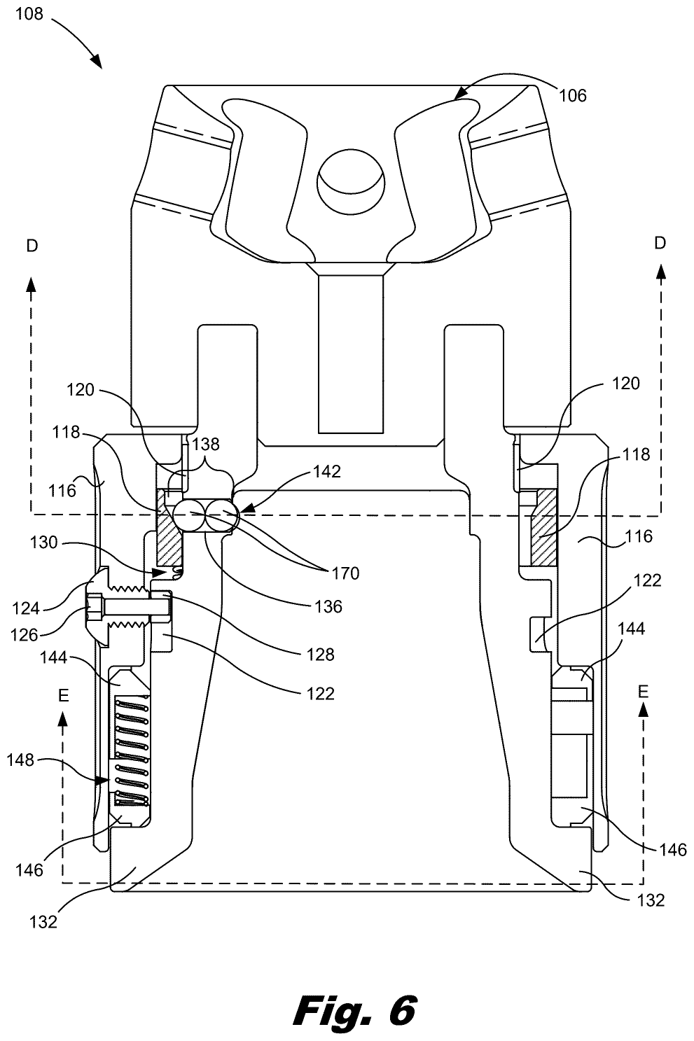

FIG. 5 is a graphic diagram side view of a prosthetic coupling device 108 without a detachable coupler of the prosthetic coupling device according to another embodiment of the present disclosure. FIG. 5 also includes a section line “C” indicating a cross-sectional plane of the prosthetic coupling device 108 as shown in FIG. 6. Additionally, FIG. 6 is a graphic diagram, side, cross-section view of a rotatable barrel 116 and an internal mount 132 of the prosthetic coupling device 108 without a detachable coupler (e.g., FIG. 4, 140) of the prosthetic coupling device 108 according to another embodiment of the present disclosure.

FIG. 5 shows the prosthetic coupling device 108 being uncoupled from the upper pylon/proximate shaft portion (not shown) at the proximal end of the prosthetic coupling device 108. Additionally, because the detachable coupler has been removed in both FIGS. 5 and 6, the prosthetic coupling device 108 in FIG. 5 is also shown to be uncoupled from the lower pylon/distal shaft portion (not shown) at a distal end of the prosthetic coupling device 108. In an embodiment, the upper socket coupler 106 of the prosthetic coupling device 108 is used to operatively couple the upper pylon/proximate shaft portion to the prosthetic coupling device 108.

In an embodiment, the upper socket coupler 106 may include socket coupler coupling holes 154. Each of the socket coupler coupling holes 154 may be used to receive a coupling device (not shown) such as a bolt, screw, set screw, or other coupling device in order to secure the upper pylon/proximate shaft portion to the proximal end of the prosthetic coupling device 108. It is appreciated that any type of coupling device may be used to secure the upper pylon/proximate shaft portion to the proximal end of the prosthetic coupling device 108 and the present specification contemplates these other coupling devices. In an embodiment, the coupling device used to secure the upper pylon/proximate shaft portion 104 to the prosthetic coupling device 108 may be secured into the socket coupler coupling holes 154 such that a patient may not be capable of removing the coupling devices thereby preventing dismantling of the upper pylon/proximate shaft portion from the prosthetic coupling device 108 during use. In an embodiment, the coupling devices may only be removed using a proprietary tool that interfaces with the coupling devices such that a patient would be incapable of manipulating these coupling devices thereby preventing the removal of the upper pylon/proximate shaft portion 104 from the prosthetic coupling device 108. This prevents a patient from altering the prosthetic device apart from the prosthetic coupling device 108 without supervision from a trained orthotic or other prosthetic fitting personnel.

FIG. 5 also shows a plurality of set screws 124 formed through the rotatable barrel 116 of the prosthetic coupling device 108. Again, the set screw 124 with its bolt 126 are passed through the rotatable barrel 116 and engages with the first vertical slot formed at a first end of the horizontal portion of the bolt slot (not shown), the horizontal portion of the bolt slot, and a second vertical slot formed at a second end of the horizontal portion of the bolt slot. In an embodiment, the prosthetic coupling device 108 shown in FIGS. 5 and 6 is placed in a locked state without the detachable coupler being locked into the internal mount (not shown) of the prosthetic coupling device 108. As described herein, the indicator ring used to indicate to a patient the locked and unlocked state of the prosthetic coupling device 108 is not viewable thereby indicating to a patient that the prosthetic coupling device 108 is in a locked state.

During operation, the patient may press down on the rotatable barrel 116 to pass the bolt 126 and set screw 124 downward along a vertical slot and into the horizontal portion of the bolt slot. By pressing down on the rotatable barrel 116, the patient overcomes the spring bias from the one or more locking ring springs and one or more spring cage springs as described herein. As the patient rotates the rotatable barrel 116 in a direction (e.g., in a counterclockwise direction), the bolt 126 and set screw 124 are passed along the entire length of the horizontal portion of the bolt slot until this assembly reaches the second end of the horizontal portion of the bolt slot and to a second vertical slot formed at a second end of the horizontal portion of the bolt slot. In an embodiment, this second vertical slot formed at the second end of the horizontal portion of the bolt slot may have a vertically lower terminal end than the first vertical slot preventing the rotatable barrel 116 from returning to a maximum height as shown in FIG. 4, for example, as the prosthetic coupling device is in the locked state. When the set screw 124/bolt 126 assembly is placed within the second vertical slot of the bolt slot, the prosthetic coupling device 108 is in an unlocked state thereby allowing a patient to insert the detachable coupler into the prosthetic coupling device 108. As the detachable coupler is inserted into the prosthetic coupling device 108, the patient may then again press down on the rotatable barrel 116 and rotate the rotatable barrel 116 in the opposite direction (e.g., a counterclockwise direction) to pass the set screw 124/bolt 126 assembly down the second vertical portion of the bolt slot, along the horizontal portion of the bolt slot, and into the first vertical portion of the bolt slot at the first end of the horizontal portion of the bolt slot. These actions place the prosthetic coupling device 108 back into a locked state and, in this case, locking the detachable coupler to the remaining portions of the prosthetic coupling device 108.

Turning now to FIG. 6, and with reference to FIG. 5 as well, the prosthetic coupling device 108 includes a locking ring 118 used to push one or more ball bearings 170 of a ball bearing set 138 into one or more coupler detents 142 formed onto an outer surface of the detachable coupler 140. In an embodiment, the locking ring 118 includes one or more sloped surfaces that, as the locking ring 118 is pushed upwards and is at a specific degree of rotation about the internal mount 132, a thicker portion of the locking ring 118 forces a series of ball bearings 170 of one or more ball bearing sets 138 through their respective horizontal ball bearing shafts 136. As the ball bearings 170 are forced through the horizontal ball bearing shafts 136, a terminal ball bearing 170 of the ball bearing set 138 may extend past an inner surface of the internal mount 132 and interface with the one or more coupler detents 142 formed on the outer surface of the detachable coupler (not shown) when the detachable coupler is present. In the embodiment shown in FIG. 5, because the detachable coupler 140 is not seated within the internal mount 132, a last in the series of ball bearings 170 of the ball bearing set 138 extends a distance past an interior surface of the internal mount 132 where the detachable coupler (with its coupler detents) would otherwise be placed.

As shown in FIG. 6, the locking ring 118 is formed around the internal mount 132 and within the rotatable barrel 116. In an embodiment, the locking ring 118 is operatively coupled to the rotatable barrel 116 such that rotation of the rotatable barrel 116 causes the locking ring 118 to also rotate the same degree. In an embodiment, the locking ring 118 may be glued to an inner surface of the rotatable barrel 116. In an embodiment, the locking ring 118 may be secured to the inner surface of the rotatable barrel 116 via one or more coupling devices such as screws, bolts, and the like. In an embodiment, the locking ring 118 may be coupled to the inner surface of the rotatable barrel 116 via an engineering fit that prevents independent rotation of the locking ring 118 relative to the rotatable barrel 116. In an embodiment, this engineering fit may include an interference fit such as a press fit, a driving fit, or a forced fit. It is appreciated that any type of engineering fit between the locking ring 118 and the rotatable barrel 116 in order to prevent independent rotation of the locking ring 118 relative to the rotatable barrel 116 during operation by the patient. In an alternative embodiment, the locking ring 118 may have one or more flat surfaces formed on an external surface of the locking ring 118 that interface, mechanically, with matching flat surfaces formed on the internal surface of the rotatable barrel 116. In this embodiment, as the rotatable barrel 116 is rotated, the flat surfaces formed on the internal surface of the rotatable barrel 116 mechanically interface with the flat surfaces formed on the external surface of the locking ring 118 such that the locking ring 118 rotates with the rotation of the rotatable barrel 116.

The locking ring 118 is formed such that as the locking ring 118 rotates about the internal mount 132, the ball bearing set 138 is allowed to move radially outward through the horizontal ball bearing shafts 136 such that they disengage with the coupler detents 142 formed on the outer surface of the detachable coupler 140. In an embodiment, a width of the locking ring 118 is reduced at certain locations along the circumference of the locking ring 118 such that extra space is provided for the movement of the ball bearings 170 of the ball bearing set 138 to move into and, therefore, move radially outward within the horizontal ball bearing shafts 136. In an embodiment, the movement of the ball bearing sets 138 radially outward through the horizontal ball bearing shafts 136 does not occur until the rotation of the locking ring 118 aligns the locations along the circumference where the width of the locking ring 118 is reduced. This may coincide with the bolt slot 122 and set screw 124/bolt 126/bolt bearing 128 assembly reaching a terminal end of the bolt slot 122. In an embodiment, this may be where the second end of the horizontal portion of the bolt slot 122 is reached by the set screw 124/bolt 126/bolt bearing 128 assembly and where the bolt bearing 128 and the end of the bolt 126 is movable into the second vertical slot formed at a second end of the horizontal portion of the bolt slot 122 as described herein.

In an embodiment, the locking ring 118 interfaces with a locking ring spring 130 at a bottom surface of the locking ring 118. The locking ring spring 130 may be biased to force the locking ring 118 upwards and towards an interior surface of the rotatable barrel 116. As described herein, under certain orientations of the locking ring 118 and rotatable barrel 116, as the locking ring 118 is forced upwards by the locking ring spring 130, the ball bearings 170 of the ball bearing set 138 are shunted radially into the horizontal ball bearing shafts 136 causing the ball bearings 170 to be forced into the coupler detents of the detachable coupler (not shown) to lock the detachable coupler into the internal mount 132 as described herein.

In an embodiment, as the rotatable barrel 116 is pressed down and rotated (e.g., rotated counterclockwise) a distance by the patient, the bolt bearing 128 and terminal end of the bolt 126 formed through the set screw 124 travels within a bolt slot 122. As the patient releases pressure applied to the rotatable barrel 116, the rotatable barrel 116 may be pushed upwards by action of, at least, the locking ring spring 130. In an embodiment, the locking ring 118, when rotated, has cavities or surfaces that allow the ball bearings 170 to fall or pass into thereby allowing the ball bearings 170 of the ball bearing set 138 to move radially outwards. This disengages the ball bearing sets 138 from their respective coupler detents formed in the outer surface of the detachable coupler, and the detachable coupler may be removed from the remaining portions of the prosthetic coupling device 108.

Where the detachable coupler is removed from the remaining portion of the prosthetic coupling device 108 as shown in FIG. 6, the lower pylon/distal shaft portion (not shown) and prosthetic foot (not shown) operatively coupled to the detachable coupler is also removed thereby allowing the patient to switch out the detachable coupler/lower pylon/distal shaft portion/prosthetic foot assembly with another detachable coupler/lower pylon/distal shaft portion/prosthetic foot assembly where needed by the patient.

As described herein, the internal mount 132 includes a bolt slot 122 formed along an outer surface of the internal mount 132. In an embodiment, the bolt slot 122 may be formed generally in a horizontal direction with a first vertical slot extending above the bolt slot 122 at a first end and a second vertical slot extending above the bolt slot 122 at a second end of the bolt slot 122. The bolt slot 122 may interface with the set screw 124/bolt 126/bolt bearing 128 assembly. As described herein, the set screw 124 and bolt 126 are passed through a via formed in the rotatable barrel 116. By setting the length of the horizontal portion of the bolt slot 122, the degree of rotation of the rotatable barrel 116 is also set. Therefore, where the horizontal portion of the bolt slot 122 extends a quarter portion around the internal mount 132, the rotatable barrel 116 is allowed to only be rotated a quarter turn. It is appreciated that the length of the horizontal portion of the bolt slot 122 may be increased or decreased in order to facilitate the operation of the prosthetic coupling device 108 as described herein. In an embodiment, the length of the horizontal portion of the bolt slot 122 further sets the degree of rotation of the rotatable barrel 116 by the patient to lock or unlock the detachable coupler 140 to the prosthetic coupling device 108 as described herein.

In an embodiment, the prosthetic coupling device 108 further includes an upper spring cage 144 and a lower spring cage 146 with one or more spring cage springs 148 formed between the upper spring cage 144 and lower spring cage 146. The upper spring cage 144 and lower spring cage 146 may be, generally, in the form of a ring that each are oriented around an outer surface of the internal mount 132. In an embodiment, an upper surface of the upper spring cage 144 may be forced against a lower surface formed on an internal surface of the rotatable barrel 116 using the one or more spring cage springs 148. Additionally, a lower surface of the lower spring cage 146 may be forced against a shelf or other surface formed on the external surface of the internal mount 132 using the one or more spring cage springs 148. Consequently, the upper spring cage 144, lower spring cage 146, and one or more spring cage springs 148 are used to apply a biased force against the rotatable barrel 116 such that the rotatable barrel 116 is forced upward away from the internal mount 132. This upward biasing force against the rotatable barrel 116 by the use of the upper spring cage 144, lower spring cage 146, and one or more spring cage springs 148 is in addition to the force applied to the rotatable barrel 116 by the locking ring spring 130 that also forces the locking ring 118 upwards and against the internal surface of the rotatable barrel 116 as described herein.

As shown in FIG. 6, the set screw 124 with its bolt 126 are passed through the rotatable barrel 116 and engages with the first vertical slot formed at a first end of the horizontal portion of the bolt slot 122 with a bolt bearing 128. In this orientation, the prosthetic coupling device 108 is in a locked state. During operation, the patient may press down on the rotatable barrel 116 to pass the bolt slot 122 and set screw 124 downward along the vertical slot and into the horizontal portion of the bolt slot 122. By pressing down on the rotatable barrel 116, the patient overcomes the spring bias from the one or more locking ring springs 130 and one or more spring cage springs 148. As the patient rotates the rotatable barrel 116 in a direction (e.g., in a counterclockwise direction), the bolt 126 and bolt bearing 128 are passed along the entire length of the horizontal portion of the bolt slot 122 until this assembly reaches the second end of the horizontal portion of the bolt slot 122 and to a second vertical slot formed at a second end of the horizontal portion of the bolt slot 122. In an embodiment, this second vertical slot formed at the second end of the horizontal portion of the bolt slot 122 may have a vertically lower terminal end than the first vertical slot preventing the rotatable barrel 116 from returning to a maximum height as shown in FIG. 4 as the prosthetic coupling device is in the locked state. When the set screw 124/bolt 126 assembly is placed within the second vertical slot of the bolt slot 122, the prosthetic coupling device 108 is in an unlocked state.

In an embodiment, the set screw 124 may have a channel formed through the entire axial length of the set screw 124. This channel may include threading to receive the bolt 126 along the axial length of the set screw 124 such that the bolt 126 is formed coaxially with the set screw 124. In an embodiment, the length of the bolt 126 exceeds the length of the set screw 124 such that the bolt 126 extends past the length of the set screw 124 a distance. As shown in FIG. 6, the bolt 126 extends past the set screw 124 and past an internal surface of the rotatable barrel 116 thereby being the only part of the set screw 124/bolt 126 assembly that enters into the bolt slot 122 formed on the external surface of the internal mount 132. Additionally, as described herein in some embodiments, the bolt 126 may be fitted with a bolt bearing 128. The bolt bearing 128 may be allowed to rotate about the axis of the bolt 126 such that as the rotatable barrel 116 is rotated, the bolt bearing 128 engages with the surfaces of the bolt slot 122 to allow for the bolt 126 and bolt bearing 128 to pass along the surface of the bolt slot 122 to reduce friction between the bolt 126 and the surfaces of the bolt slot 122. Additionally, the diameter of the bolt bearing 128 may be selected such that as the bolt 126 and bolt bearing 128 reach the vertical portions of the bolt slot 122, the bolt bearing 128 fits and is seated within these vertical portions of the bolt slot 122.

In an embodiment, the prosthetic coupling device 108 may include an indicator ring 120. In an embodiment, the indicator ring 120 may be used by the patient to determine the locked or unlocked state of the prosthetic coupling device 108. As described herein, when the prosthetic coupling device 108 is in a locked state as shown in FIG. 4, the rotatable barrel 116 is in a maximum vertical position. With the rotatable barrel 116 in this maximum vertical position, the indicator ring 120 is not visible to the patient because the rotatable barrel 116 is physically covering the indicator ring 120. However, as described herein, when the prosthetic coupling device 108 is in an unlocked state, the position of the rotatable barrel 116 is altered such that the rotatable barrel 116 is at a lower position than when the prosthetic coupling device 108 is in a locked state. With the rotatable barrel 116 at a lower position, the indicator ring 120 is made visible by the patient. Therefore, in an embodiment, where the patient can see the indicator ring 120, the patient will understand that the prosthetic coupling device 108 is in an unlocked state and that the detachable coupler 140/lower pylon/distal shaft portion 112/prosthetic foot 114 assembly may be removed from the remaining portions of the prosthetic coupling device 108. Even further, where the patient sees the indicator ring 120, the patient may understand that the patient should not place weight on the prosthetic device (e.g., FIG. 1, 100) or otherwise walk using the prosthetic device due to the detachable coupler 140 not being operatively locked to the remaining portions of the prosthetic coupling device 108 as described herein.

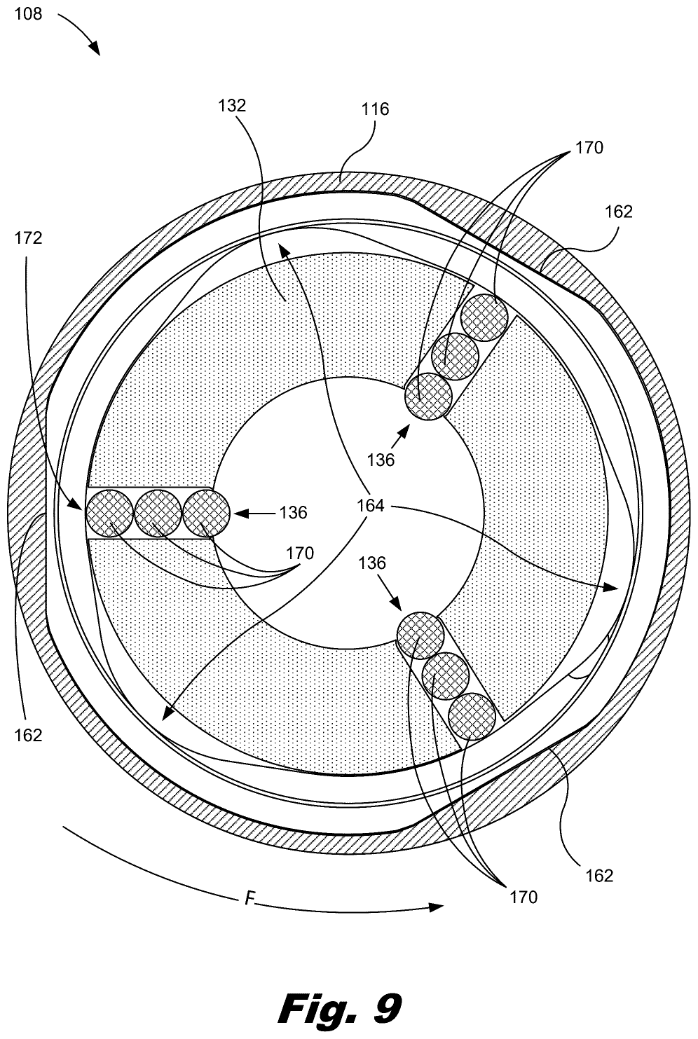

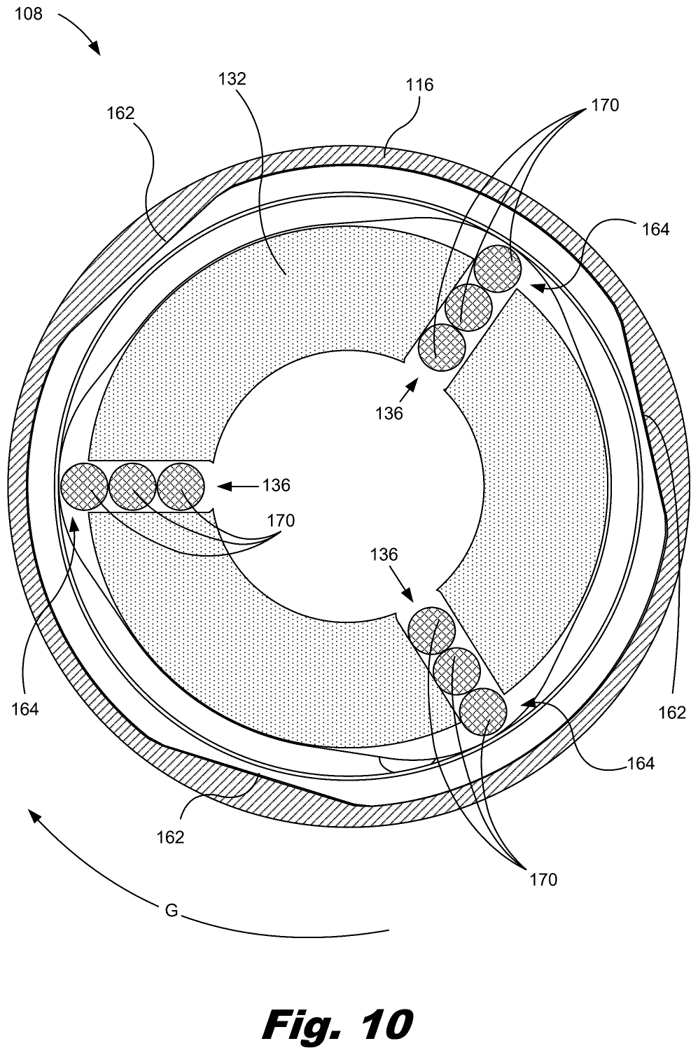

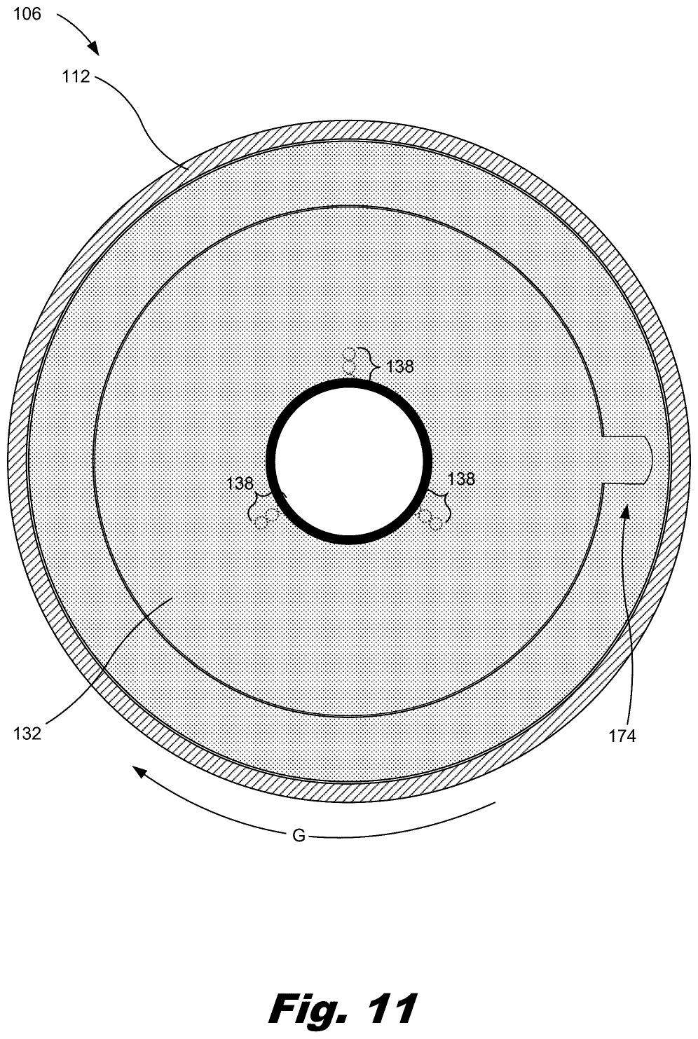

FIG. 6 also includes section lines “D” and “E.” Section line “D” indicates a cross-sectional plane of the prosthetic coupling device 108 as shown in, for example, FIGS. 9 and 10 while section line “E” indicates a cross-sectional plane of the prosthetic coupling device 108 as shown in, for example, FIG. 11. These figures will be described in more detail herein.

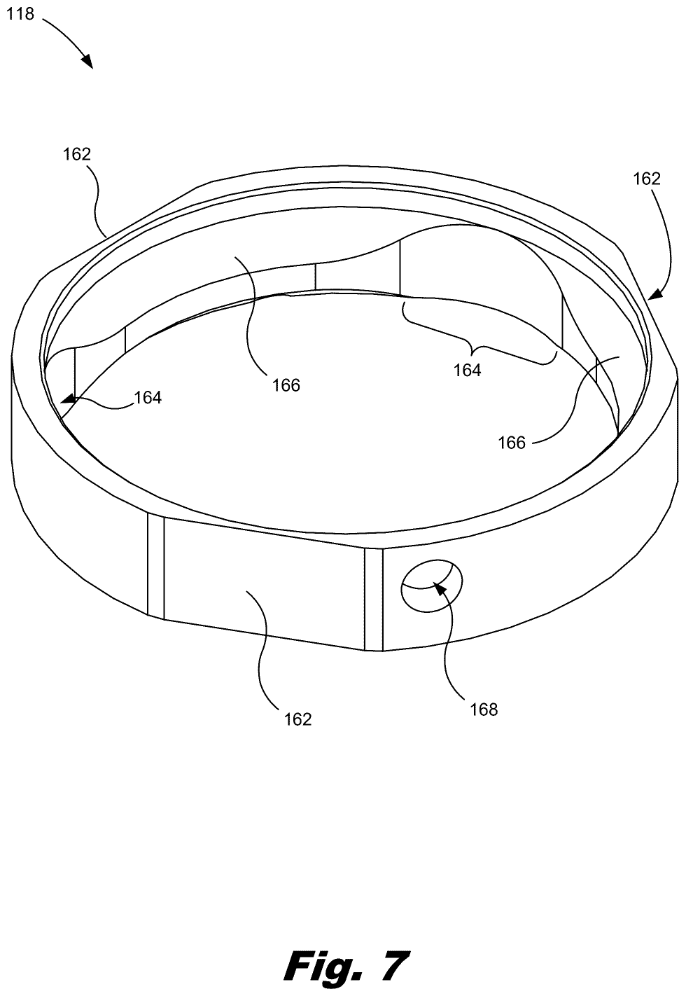

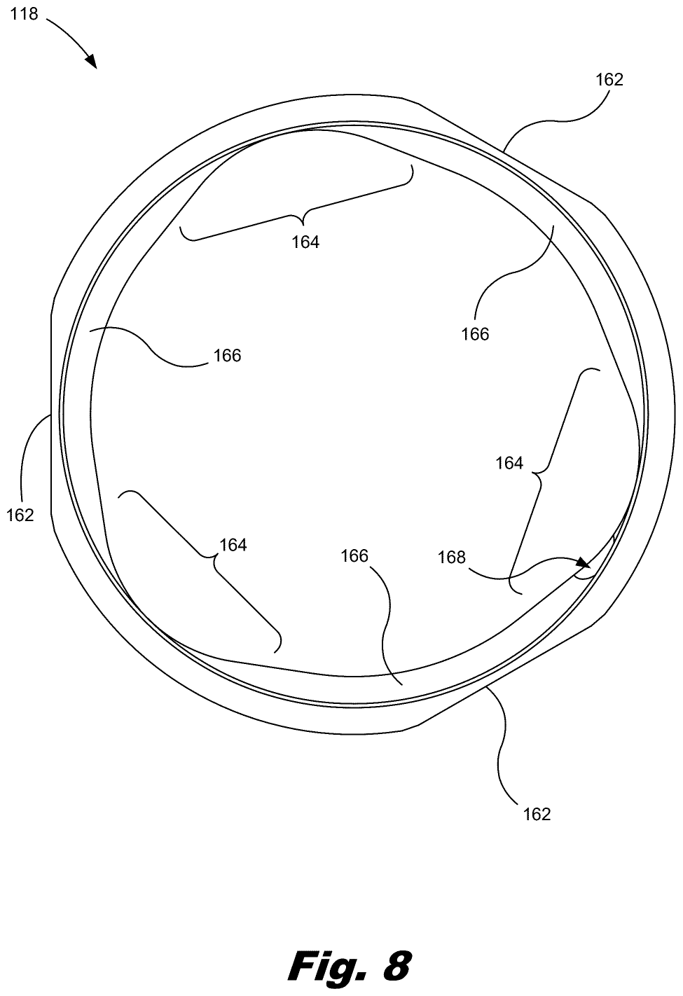

FIG. 7 is a graphic diagram front, perspective view of a locking ring 118 of the prosthetic coupling device 108 according to an embodiment of the present disclosure. Additionally, FIG. 8 is a graphic diagram top view of the locking ring 118 of the prosthetic coupling device 108 according to another embodiment of the present disclosure. As described herein, the locking ring 118 is used to shunt or otherwise push ball bearings (not shown) of one or more ball bearing sets 138 radially inward to engage with one or more coupler detents (not shown) formed in an exterior surface of the detachable coupler (not shown).

Turning first to FIG. 7, the locking ring 118 includes a plurality of barrel interlocking surfaces 162. The barrel interlocking surfaces 162 are formed on an exterior surface of the locking ring 118. As described herein, the barrel interlocking surfaces 162 interface with complementary locking surfaces formed on the internal surface of the rotatable barrel (not shown). The mechanical engagement between the barrel interlocking surfaces 162 and those complementary locking surfaces formed on the internal surface of the rotatable barrel causes the locking ring 118 to rotate with the rotatable barrel during operation by the patient. As described herein, an engineering fit may be used between the outer surface of the locking ring 118 and the internal surface of the rotatable barrel so that the locking ring 118 does not move within the rotatable barrel. In an embodiment, the locking ring 118 may be coupled to the inner surface of the rotatable barrel via an engineering fit that prevents independent rotation of the locking ring 118 relative to the rotatable barrel and/or prevents the locking ring 118 from moving vertically independent from the vertical movement of the rotatable barrel. In an embodiment, this engineering fit may include an interference fit such as a press fit, a driving fit, or a forced fit. It is appreciated that any type of engineering fit between the locking ring 118 and the rotatable barrel in order to prevent independent rotation of the locking ring 118 relative to the rotatable barrel during operation by the patient. In an alternative embodiment, the locking ring 118 may have one or more flat surfaces formed on an external surface of the locking ring 118 that interface, mechanically, with matching flat surfaces formed on the internal surface of the rotatable barrel.

In an embodiment, the locking ring 118 further includes a plurality of ball bearing recesses 164. As described herein, these ball bearing recesses 164 may allow the ball bearings of the ball bearing sets to move radially outward from a central axis of the prosthetic coupling device. This radial outward movement of the ball bearings allows the decoupling of the detachable coupler from the remaining portions of the prosthetic coupling device by disengaging a terminal ball bearing within the series of ball bearings in the ball bearing sets from the coupler detents formed on the outer surface of the detachable coupler. In an embodiment, the ball bearing recesses 164 may allow a portion of only a single ball bearing in the ball bearing sets to enter into the ball bearing recesses 164 so that a portion of the ball bearing still remains within their respective horizontal ball bearing shafts. As described herein, as the patient rotates the rotatable barrel and the locking ring 118, the ball bearing recesses 164 may be arranged at the ends of the horizontal ball bearing shafts thereby allowing the ball bearings to pass radially outward within the horizontal ball bearing shafts to allow the decoupling of the detachable coupler as described herein.

Also shown in FIGS. 7 and 8, the locking ring 118 includes one or more sloped internal surfaces 166. As described herein, the one or more sloped internal surfaces 166, as the locking ring 118 is pushed upwards and is at a specific degree of rotation about the internal mount 132, causes a thicker portion of the locking ring 118 to force the series of ball bearings of the one or more ball bearing sets through their respective horizontal ball bearing shafts. As the ball bearings are forced through their respective horizontal ball bearing shafts, a terminal ball bearing of the ball bearing set may extend past an inner surface of the internal mount and interface with the one or more coupler detents formed on the outer surface of the detachable coupler. In an embodiment, a locking ring spring (not shown) may be placed under the locking ring 118 and biased to force the locking ring 118 upwards and towards an interior surface of the rotatable barrel so as to force the ball bearing through the horizontal ball bearing shafts as described.

In an embodiment, the locking ring 118 includes a ball bearing feed hole 168. As shown in both FIGS. 7 and 8, the ball bearing feed hole 168 may be used to introduce the ball bearings into their respective horizontal ball bearing shafts during manufacturing and assembly of the prosthetic coupling device. During assembly, a manufacturer may pass a sufficient number of ball bearings through the ball bearing feed hole 168 such that the space within the horizontal ball bearing shafts is filled with ball bearings. As described herein, the ball bearings may be of a sufficient diameter such that their combined diameters fill up their respective horizontal ball bearing shafts for a portion of a terminal ball bearing to extend past, slightly, an internal surface of the internal mount or extend, slightly, into a respective ball bearing recess 164 aligned with the horizontal ball bearing shafts. Once a sufficient number of ball bearings have been introduced through the ball bearing feed hole 168 and into their respective horizontal ball bearing shafts, the ball bearing feed hole 168 may be capped off and the rotatable barrel may be slipped over the locking ring 118 thereby preventing the ball bearings from leaving their respective horizontal ball bearing shafts.

Turning now to FIG. 9, FIG. 9 is a graphic diagram, bottom, cross-section view of the rotatable barrel, locking ring, and an internal mount of the prosthetic coupling device according to an embodiment of the present disclosure. Additionally, FIG. 10 is a graphic diagram, bottom, cross-section view of the rotatable barrel, locking ring, and an internal mount of the prosthetic coupling device according to another embodiment of the present disclosure. As previously mentioned herein, FIGS. 9 and 10 are graphic diagrams of a cross-sectional plane of the prosthetic coupling device as defined by section line “D” in FIG. 6. FIGS. 9 and 10 show the engagement of the locking ring 118 with the rotatable barrel 116 as well as the interactions of the locking ring 118 with the ball bearings 170 of the ball bearing sets 138 within their horizontal ball bearing shafts 136.

Turning first to FIG. 9, the prosthetic coupling device 108 is shown to be in a locked orientation without the detachable coupler present as shown in FIG. 6. It is appreciated that the prosthetic coupling device 108 may be placed into a locked orientation or state even without the detachable coupler being present.

As shown in FIG. 9, three ball bearing engagement surfaces 172 are abutting an end ball bearing 170 among three ball bearing sets 138 such that the ball bearing engagement surfaces 172 have pushed their respective ball bearing sets 138 radially inward through the respective horizontal ball bearing shafts 136. It is appreciated that that although FIGS. 9 and 10 show three horizontal ball bearing shafts 136 with a ball bearing set 138 having three ball bearings 170, the number of horizontal ball bearing shafts 136, ball bearing sets 138, and ball bearings 170 within those sets 138 may vary depending on the number of coupler detents (not shown) formed in to the outer surface of the detachable coupler (not shown). Thus, the present specification contemplates that more or fewer horizontal ball bearing shafts 136, ball bearing sets 138, and ball bearings 170 may be used.

In this locked state, the patient has rotated the rotatable barrel 116 in a clockwise direction. However, because FIGS. 9 and 10 show a bottom view of the prosthetic coupling device 108, the patient has rotated the rotatable barrel 116 in a direction indicated by arrow “F” which is a counter-clockwise direction from this bottom view. As described herein, the shunting of the ball bearings 170 of the ball bearing sets 138 through the horizontal ball bearing shafts 136 causes a terminal ball bearing 170 to extend past an outer surface of the internal mount 132. The terminal ball bearings 170, however, are prevented from leaving the horizontal ball bearing shafts 136 due to a necking of the horizontal ball bearing shafts 136. In an embodiment, this necked portion of the horizontal ball bearing shafts 136 may have a diameter that is slightly smaller than the diameter of the ball bearings 170 such that a portion of the terminal ball bearings 170 may stick out from the internal surface of the internal mount 132. As described herein, this extension of the terminal ball bearing 170 out from the internal surface of the internal mount 132 allows the terminal ball bearings 170 to enter into or otherwise engage with respective coupler detents formed on the outer surface of the detachable coupler.

The orientation of the locking ring 118 shown in FIG. 9 shows that the ball bearing recesses 164 are not aligned, radially with the horizontal ball bearing shafts 136. Thus, the ball bearing engagement surfaces 172 next to the ball bearing recesses 164 prevent the ball bearings 170 of the ball bearing sets 138 from moving outward, radially, within the horizontal ball bearing shafts 136 such that the prosthetic coupling device 108 remains in a locked state or configuration. It is appreciated that when the rotatable barrel 116 and locking ring 118 are in this orientation shown in FIG. 9, the set screw/bolt/bolt bearing assembly (not shown) has been moved along the bolt slot such that it has reached a first end of the bolt slot and has been placed within a first vertical slot of the bolt slot. With the placement of the set screw/bolt/bolt bearing assembly within the first vertical slot of the bolt slot, the prosthetic coupling device 108 remains in this locked state until the patient presses down on the rotatable barrel 116 and rotates the rotatable barrel 116 in a counter-clockwise direction as viewed by the patient (a clockwise direction relative to FIG. 9).

FIG. 10 shows the prosthetic coupling device 108 in an unlocked position. As indicated by arrow “G,” the user has rotated the rotatable barrel 116 and locking ring 118 in a clockwise direction (e.g., a counter-clockwise direction from the view of the patient). Again, because the locking ring 118 has barrel interlocking surfaces 162 that interface with complementary surfaces formed on the interior surfaces of the rotatable barrel 116, the locking ring 118 is rotated along with the rotatable barrel 116.

At this unlocked position shown in FIG. 10, the ball bearing recesses 164 of the locking ring 118 are aligned with their respective horizontal ball bearing shafts 136. This orientation of the rotatable barrel 116 and locking ring 118 allows the end ball bearings 170 of the ball bearing set 138 to extend, slightly, past an external surface of the internal mount 132. This allows the terminal ball bearing of the ball bearing set 138 to be shunted or moved radially outward from the axis of the prosthetic coupling device 108 and towards the rotatable barrel 116 thereby not extending past an internal surface of the internal mount 132. Because the terminal ball bearing 170 of the ball bearing set 138 cannot engage with the coupler detents of the detachable coupler, the detachable coupler is removable from the prosthetic coupling device 108.

It is appreciated that when the rotatable barrel 116 and locking ring 118 are in this unlocked orientation shown in FIG. 10, the set screw/bolt/bolt bearing assembly (not shown) has been moved along the bolt slot such that it has reached a second end of the bolt slot and has been placed within a second vertical slot of the bolt slot. With the placement of the set screw/bolt/bolt bearing assembly within the second vertical slot of the bolt slot, the prosthetic coupling device 108 remains in this unlocked state until the patient presses down on the rotatable barrel 116 and rotates the rotatable barrel 116 in a clockwise direction as viewed by the patient (a counter-clockwise direction relative to FIG. 10).

FIG. 11 is a graphic diagram, bottom, cross-section view of the rotatable barrel 116, and an internal mount 132 of the prosthetic coupling device 108 according to an embodiment of the present disclosure. As indicated herein, FIG. 11 is a cross-sectional plane of the prosthetic coupling device 108 as shown in FIG. 6 at section line “E.” This cross-sectional view shows the internal mount 132 and rotatable barrel 116 with, in this example embodiment, the prosthetic coupling device 108 in an unlocked orientation as indicated by the positioning of the ball bearings of the ball bearing sets 138 shown in ghost. Additionally, FIG. 11 shows line “G” similar to that shown in FIG. 10 indicating that the patient has rotated the rotatable barrel 116 in a clockwise direction (e.g., a counter-clockwise direction as viewed from the patient's perspective).

FIG. 11 also shows a registering slot 174 formed into a portion of the internal mount 132. As described herein, the detachable coupler (not shown) that mates with the internal mount 132 further includes, in an embodiment, a registering post (not shown) formed on an outside surface of the detachable coupler (not shown). The registering post is used by the patient to properly align the detachable coupler relative to the internal mount 132 of the prosthetic coupling device 108. In an embodiment, the registering post interfaces with this registering slot 174 formed into a bottom edge of the internal mount 132 of the prosthetic coupling device 108. This alignment of the detachable coupler relative to the internal mount 132 prevents the patient from misaligning the lower pylon and foot (or another prosthetic terminal device) relative to the upper pylon described in FIG. 1. Once aligned, the patient may lock the detachable coupler to the internal mount 132 of the prosthetic coupling device 108 as described herein (e.g., rotating the rotatable barrel 116) in order to be able place weight on the prosthetic coupling device 108/prosthetic device and walk properly using the prosthetic device.

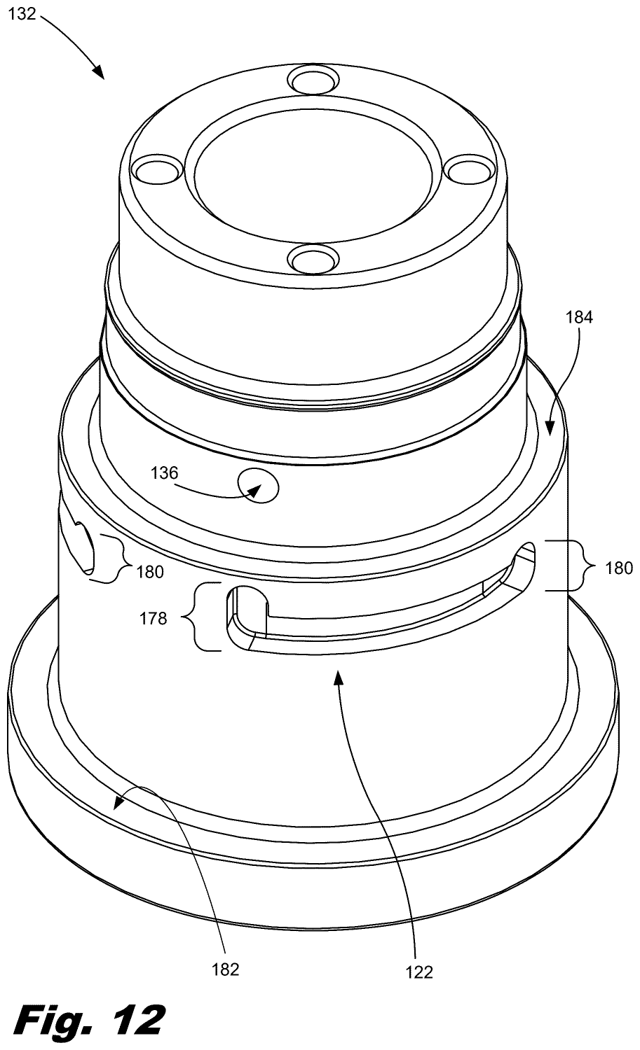

FIG. 12 is a graphic diagram, side, perspective view of an internal mount 132 of the prosthetic coupling device according to an embodiment of the present disclosure. As described herein, the internal mount 132 may interface with a locking ring (not shown) and rotatable barrel (not shown) to selectively unlock and lock a detachable coupler (not shown) to the prosthetic coupling device.

As described herein, the internal mount 132 includes a bolt slot 122 formed onto an exterior surface of the internal mount 132. This internal mount 132 includes a, generally, horizontal section. At a first end of the horizontal section of the bolt slot 122, a first vertical slot 178 is formed. A second vertical slot 180 is also formed at a second end of the horizontal section of the bolt slot 122. As described herein, the set screw with its bolt are passed through the rotatable barrel and engages with the first vertical slot 178 formed at the first end of the horizontal portion of the bolt slot 122. When the bolt with its bolt bearing is at the first vertical slot 178, the prosthetic coupling device is in a locked state with the detachable coupler being locked into the internal mount 132 of the prosthetic coupling device in an embodiment. During operation, the patient may press down on the rotatable barrel to pass the bolt and set screw downward along the vertical slot and into the horizontal portion of the bolt slot 122. By pressing down on the rotatable barrel, the patient overcomes the spring bias from the one or more locking ring springs (not shown) and one or more spring cage springs (not shown) as described herein. As the patient rotates the rotatable barrel in a direction (e.g., in a counterclockwise direction), the bolt and a bolt bearing formed on the end of the bolt are passed along the entire length of the horizontal portion of the bolt slot until this assembly reaches the second end of the horizontal portion of the bolt slot and to the second vertical slot 180 formed at the second end of the horizontal portion of the bolt slot 122. In an embodiment, this second vertical slot 180 formed at the second end of the horizontal portion of the bolt slot 122 may have a vertically lower terminal end than the first vertical slot 178 preventing the rotatable barrel from returning to a maximum height (e.g., as shown in FIG. 4) as the prosthetic coupling device is in the locked state. When the set screw/bolt assembly is placed within the second vertical slot 180 of the bolt slot 122, the prosthetic coupling device is in an unlocked state.

The internal mount 132 also includes one or more (e.g., three) horizontal ball bearing shafts 136. As described herein, these horizontal ball bearing shafts 136 are used to house ball bearings (not shown) of the ball bearing set. These ball bearings may interact with the locking ring as described herein in order to selectively move the ball bearings radially inward and outward though the horizontal ball bearing shafts 136. As described herein, the locking ring is forced upward via a locking ring spring. In order to create this biasing force, the internal mount 132 includes a locking ring spring shelf 184 onto which the locking ring spring may push off from in order to force the locking ring upwards.

As described herein, the prosthetic coupling device includes a lower spring cage (not shown) that is placed around the internal mount 132. In an embodiment, the lower spring cage may interact with the upper spring cage and spring cage spring to apply an upward force against the rotatable barrel as described herein. In an embodiment, the lower spring cage may rest on a lower spring cage shelf 182 so that the spring cage spring placed between the lower spring cage and upper spring cage may apply this upward force against the rotatable barrel.

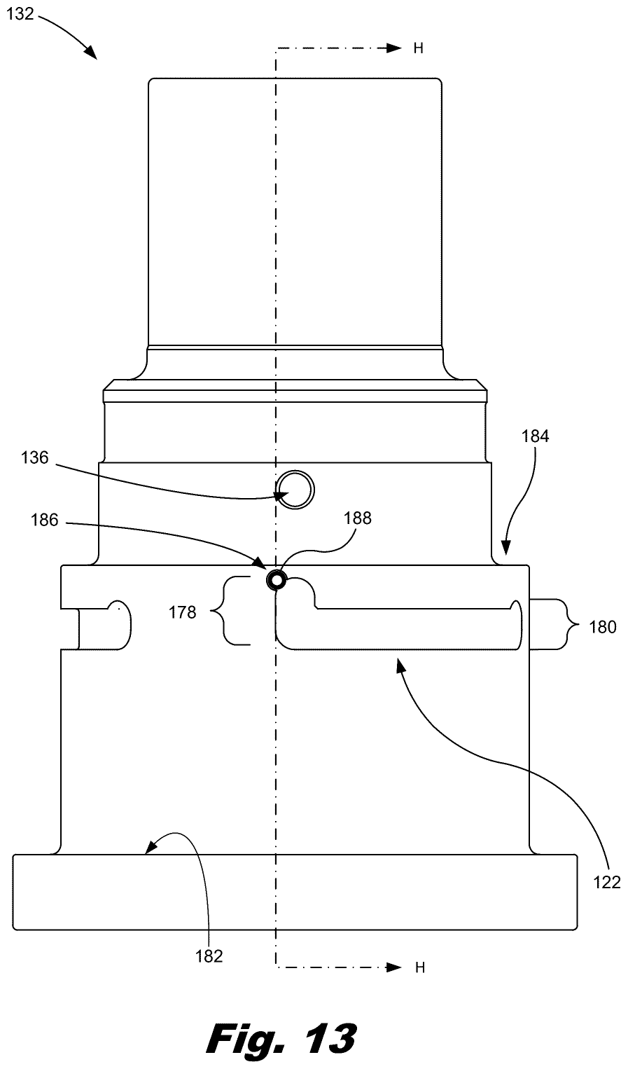

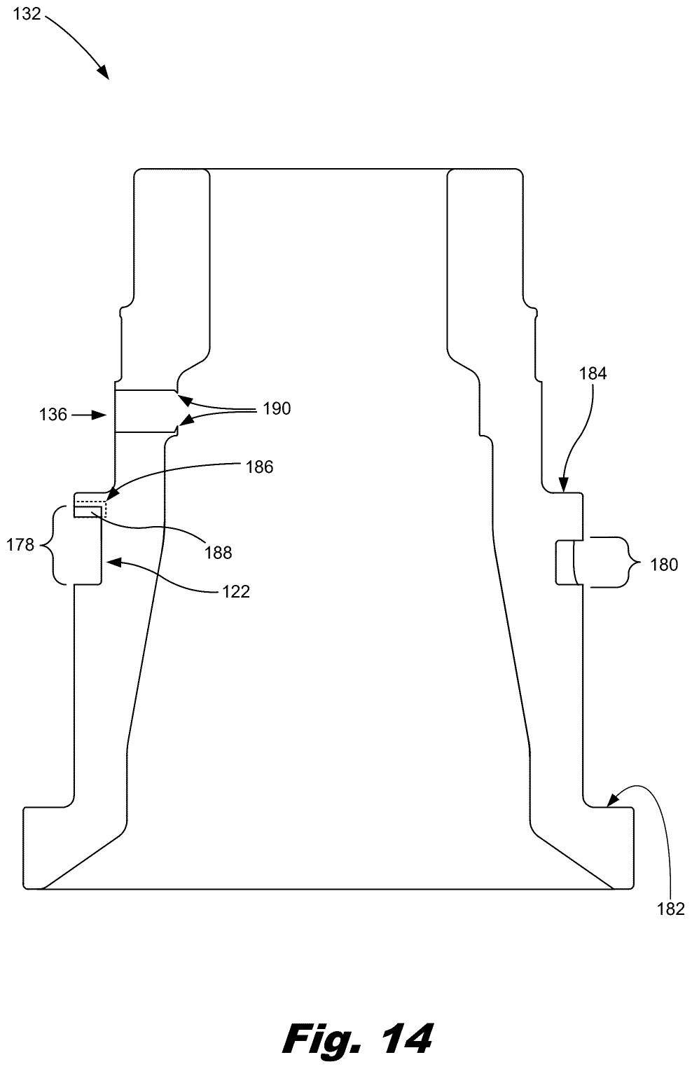

FIG. 13 is a graphic diagram, side view of an internal mount of the prosthetic coupling device according to another embodiment of the present disclosure. FIG. 13 also includes a section line “H” indicating a cross-sectional plane of the internal mount 132 as shown in FIG. 13. Accordingly, FIG. 14 is a graphic diagram side, cross-sectional view of an internal mount 132 of the prosthetic coupling device according to another embodiment of the present disclosure.

Again, the internal mount 132 includes a bolt slot 122 formed onto an exterior surface of the internal mount 132. This internal mount 132 includes a, generally, horizontal section. At a first end of the horizontal section of the bolt slot 122, a first vertical slot 178 is formed. A second vertical slot 180 is also formed at a second end of the horizontal section of the bolt slot 122. As described herein, the set screw with its bolt are passed through the rotatable barrel and engages with the first vertical slot 178 formed at the first end of the horizontal portion of the bolt slot 122. When the bolt with its bolt bearing is at the first vertical slot 178, the prosthetic coupling device is in a locked state with the detachable coupler being locked into the internal mount 132 of the prosthetic coupling device in an embodiment. During operation, the patient may press down on the rotatable barrel to pass the bolt and set screw downward along the vertical slot and into the horizontal portion of the bolt slot 122. By pressing down on the rotatable barrel, the patient overcomes the spring bias from the one or more locking ring springs (not shown) and one or more spring cage springs (not shown) as described herein. As the patient rotates the rotatable barrel in a direction (e.g., in a counterclockwise direction), the bolt and a bolt bearing formed on the end of the bolt are passed along the entire length of the horizontal portion of the bolt slot until this assembly reaches the second end of the horizontal portion of the bolt slot and to the second vertical slot 180 formed at the second end of the horizontal portion of the bolt slot 122. In an embodiment, this second vertical slot 180 formed at the second end of the horizontal portion of the bolt slot 122 may have a vertically lower terminal end than the first vertical slot 178 preventing the rotatable barrel from returning to a maximum height (e.g., as shown in FIG. 4) as the prosthetic coupling device is in the locked state. When the set screw/bolt assembly is placed within the second vertical slot 180 of the bolt slot 122, the prosthetic coupling device is in an unlocked state.

The internal mount 132 also includes one or more (e.g., three) horizontal ball bearing shafts 136. As described herein, these horizontal ball bearing shafts 136 are used to house ball bearings (not shown) of the ball bearing set. These ball bearings may interact with the locking ring as described herein in order to selectively move the ball bearings radially inward and outward though the horizontal ball bearing shafts 136. As described herein, the locking ring is forced upward via a locking ring spring. In order to create this biasing force, the internal mount 132 includes a locking ring spring shelf 184 onto which the locking ring spring may push off from in order to force the locking ring upwards.

As described herein, the prosthetic coupling device includes a lower spring cage (not shown) that is placed around the internal mount 132. In an embodiment, the lower spring cage may interact with the upper spring cage and spring cage spring to apply an upward force against the rotatable barrel as described herein. In an embodiment, the lower spring cage may rest on a lower spring cage shelf 182 so that the spring cage spring placed between the lower spring cage and upper spring cage may apply this upward force against the rotatable barrel.

The internal mount 132 shown in FIG. 13 also includes a first vertical slot detent 186 formed at a terminal end of the first vertical slot 178. The first vertical slot detent 186 includes a rubber gasket 188 placed therein. The first vertical slot detent 186 with its rubber gasket 188 may be formed to prevent the bolt and bolt bearing from rattling against the surfaces of the bolt slot 122 when the prosthetic device is in a locked state. In an embodiment, as the patient uses the prosthetic device, the force applied by the locking ring springs and spring cage springs may be overcome when the prosthetic coupling device is subjected to impacts such as when the patient is walking or running using the prosthetic device. The rubber gasket 188 placed in the first vertical slot detent 186 may dampen or eliminate the noise created by impacts of the bolt and bolt bearing within the first vertical slot 178. In an embodiment, the rubber gasket 188 may be operatively coupled within the first vertical slot detent 186 using a glue or mechanical fastener. In an embodiment, the rubber gasket 188 may be forced into the first vertical slot detent 156 via an engineering fit such that the rubber gasket 188 does not move within the first vertical slot detent 186. In an embodiment, the rubber gasket 188 may generally be in a columnar shape. In an embodiment, the rubber gasket 188 may be in a spherical shape.

FIG. 14 shows additional features of the internal mount 132 including one or more ball bearing shaft lips 190. The ball bearing shaft lips 190 are used to prevent the ball bearings that are placed into the horizontal ball bearing shafts 136 from moving past the internal surface of the internal mount 132. In an embodiment, these ball bearing shaft lips 190 may neck or otherwise reduce the diameter of the horizontal ball bearing shafts 136 smaller than the diameter of the ball bearings. The ball bearing shaft lips 190, however, allow a portion of a leading ball bearing of the ball bearing set to extend out of the horizontal ball bearing shafts 136 to engage with the coupler detents of the detachable coupler as described in embodiments herein.

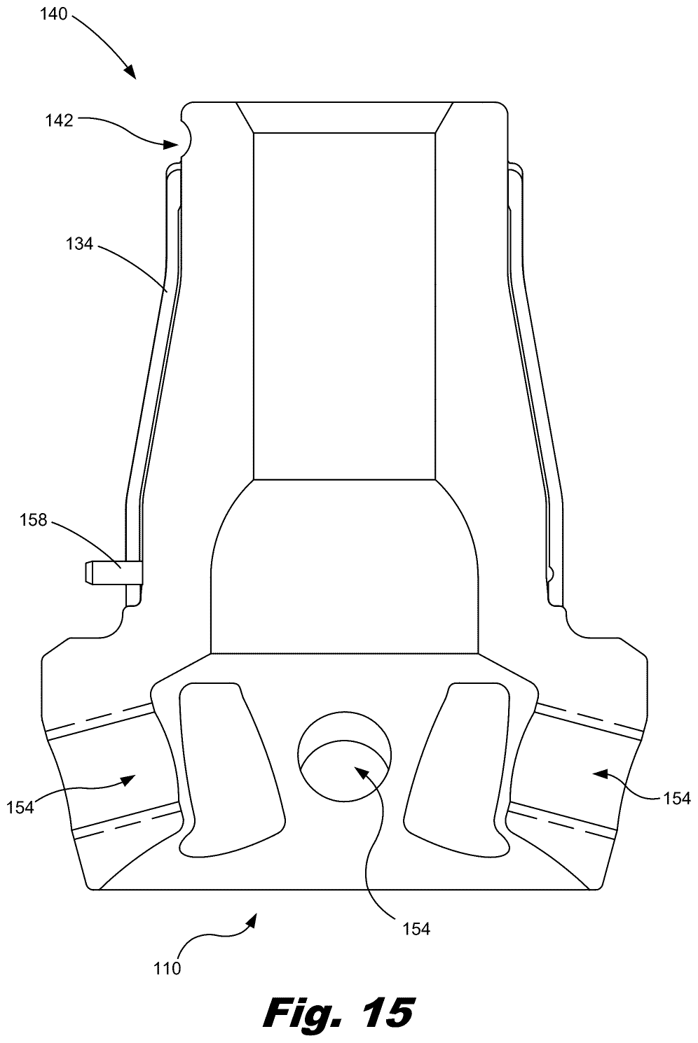

FIG. 15 is a graphic diagram side, cross-section view of a detachable coupler 140 of the prosthetic coupling device according to another embodiment of the present disclosure. FIG. 15 is a cross section of the detachable coupler 140 as defined by section line “B” in FIG. 3. As described herein, the detachable coupler 140 may include the lower socket coupler 110. The lower socket coupler 110 may include socket coupler coupling holes 154. Each of the socket coupler coupling holes 154 may be used to receive a coupling device (not shown) such as a bolt, screw, set screw, or other coupling device in order to secure the lower pylon/distal shaft portion (not shown) to the distal end of the prosthetic coupling device. It is appreciated that any type of coupling device may be used to secure the lower pylon/distal shaft portion to the distal end of the prosthetic coupling device and the present specification contemplates these other coupling devices. In an embodiment, the coupling device used to secure the lower pylon/distal shaft portion to the prosthetic coupling device may be secured into the socket coupler coupling holes 154 such that a patient may not be capable of removing the coupling devices thereby preventing dismantling of the lower pylon/distal shaft portion from the prosthetic coupling device during use. In an embodiment, the coupling devices may only be removed using a proprietary tool that interfaces with the coupling devices such that a patient would be incapable of manipulating these coupling devices thereby preventing the removal of the lower pylon/distal shaft portion from the prosthetic coupling device. This prevents a patient from altering the prosthetic device apart from the prosthetic coupling device without supervision from a trained orthotic or other prosthetic fitting personnel.

In an embodiment, the detachable coupler 140 may include an outer plastic sleeve 134 formed on an outer surface of the detachable coupler 140. In an embodiment, the outer plastic sleeve 134 may be made of an acetal plastic or other similar material that has a low surface energy and that has a relatively high wear resistance and high strength. The low surface energy of the outer plastic sleeve 134 allows the detachable coupler 140 to be easily slid into and out of the internal mount 132 of the prosthetic coupling device 108 during operation by the patient. In an embodiment, this outer plastic sleeve 134 may be milled to fit around the outer surface of the detachable coupler 140. In an embodiment, the outer plastic sleeve 134 may be injection molded onto the detachable coupler 140.

The high strength and wear resistance of the outer plastic sleeve 134 allows for the multiple coupling and uncoupling of the detachable coupler 140 from the internal mount 132 of the prosthetic coupling device 108 with reduced amounts of wear and tear between the detachable coupler 140 and the internal mount 132. In another embodiment, the outer plastic sleeve 134 may be coupled to the outer surface of the detachable coupler 140 via a glue or other adhesive. In an embodiment, the glue or adhesive may include RelTek® Bondit® B-4682TH or other similar adhesives that adhere the outer plastic sleeve 134 to the metal (e.g. aluminum such as 2024-T351 aluminum and similar alloys) of the detachable coupler 140.

The inclusion of the outer plastic sleeve 134 acts as a lubricant, facilitating easy separation of detachable coupler 140 from the internal mount 132 of the prosthetic coupling device 108 for easier use by the patient. Additionally, it is appreciated that other types of plastics or materials different from the acetal plastic of the outer plastic sleeve 134 may be used and the present specification contemplates the use of these other materials. In an embodiment, for example, a brass sleeve 134 could also provide similar properties as the acetal plastic of the outer plastic sleeve 134. It is appreciated that surface treatments other than the outer plastic sleeve 134 may be placed over the internal mount 132 to provide a low friction surface between the internal mount 132 of the detachable coupler 140 as well as any other internal surfaces within the internal mount 132.

As described herein, the detachable coupler 140 mates and is aligned with the internal mount of the prosthetic device via a registering post 158 formed on an outside surface of the detachable coupler 140. The registering post is used by the patient to properly align the detachable coupler relative to the internal mount of the prosthetic coupling device by the registering post 158 fitting into a registering slot (e.g., FIG. 11, 174) formed into the internal mount as described herein. In an embodiment, the registering post 158 interfaces with this registering slot formed into a bottom edge of the internal mount of the prosthetic coupling device. This alignment of the detachable coupler 140 relative to the internal mount prevents the patient from misaligning the lower pylon and foot (or another prosthetic terminal device) relative to the upper pylon described in FIG. 1. Once aligned, the patient may lock the detachable coupler 140 to the internal mount of the prosthetic coupling device as described herein (e.g., rotating the rotatable barrel 116) in order to be able place weight on the prosthetic coupling device/prosthetic device and walk properly using the prosthetic device.

Additionally, the detachable coupler 140 shown in FIG. 15 includes one or more coupler detents 142. As described herein, the number and radial position of each of the coupler detents 142 formed into the detachable coupler 140 may match the number and radial position of the horizontal ball bearing shaft (not shown) and ball bearing sets (not shown) that are formed into the internal mount of the prosthetic coupling device described herein. As described herein, the coupler detents 142 may be a detent that forms a location where a terminal ball bearing of the ball bearing set may be seated. In an embodiment, the terminal ball bearing of the ball bearing set is seated into the coupler detent 142 as the detachable coupler 140 is seated into the internal mount of the prosthetic coupling device and the patient has rotated the rotatable barrel (not shown) as described herein.

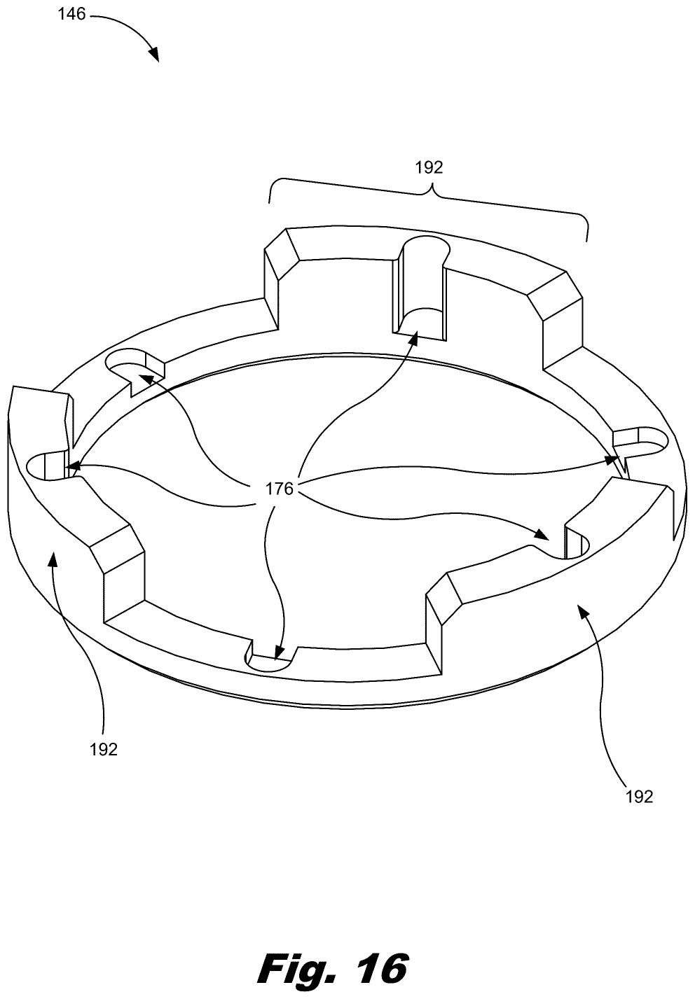

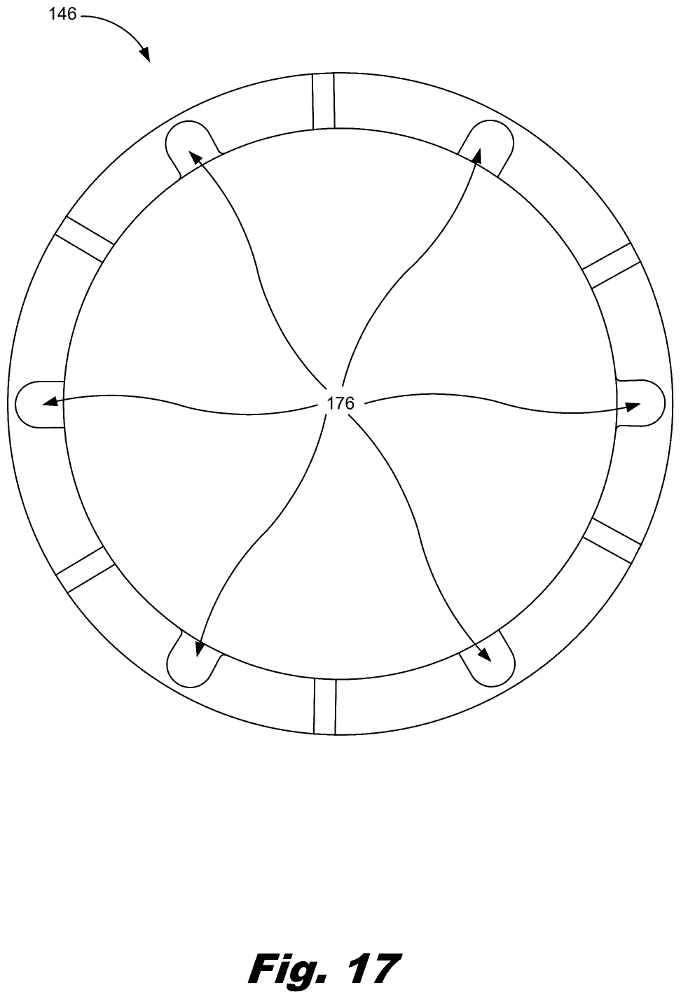

FIG. 16 is graphic diagram side, perspective view of a lower spring cage 146 of the prosthetic coupling device according to another embodiment of the present disclosure. Additionally, FIG. 17 is a graphic diagram top view of the lower spring cage according to another embodiment of the present disclosure.

FIGS. 16 and 17 show the lower spring cage 146 including a plurality of spring cage spring recesses 176. The spring cage spring recesses 176 are each formed to receive a spring cage spring (not shown). As described herein, the prosthetic coupling device may include both an upper spring cage and the lower spring cage 146 with one or more spring cage springs formed between the upper spring cage and lower spring cage 146. In an embodiment, each of the upper spring cage and lower spring cage 146 may be made of a plastic such as an acetal plastic. In an embodiment, the upper spring cage and lower spring cage 146 may be made of low friction material that allows moving elements of the prosthetic coupling device such as the rotatable barrel to move easily over the surfaces of the upper spring cage and lower spring cage 146. In an embodiment, the upper spring cage and lower spring cage 146 may be mechanically affixed to an outer surface of the internal mount such that the upper spring cage and lower spring cage 146 do not rotate with the rotation of the rotatable barrel, for example. In these embodiments, the upper spring cage and lower spring cage 146 may be operatively coupled to the external surface of the internal mount using a coupling device (bolt, screw, nail, etc.), a glue, or via an engineering fit that prevents movement of the upper spring cage and lower spring cage 146 relative to the internal mount.

As shown in FIGS. 16 and 17, the lower spring cage 146 may be, generally, in the form of a ring that is oriented around an outer surface of the internal mount. In an embodiment, an upper surface of the upper spring cage may be forced against a lower surface formed on an internal surface of the rotatable barrel using the one or more spring cage springs. Additionally, a lower surface of the lower spring cage 146 may be forced against a shelf or other surface formed on the external surface of the internal mount using the one or more spring cage springs. Consequently, the upper spring cage, lower spring cage 146, and one or more spring cage springs are used to apply a biased force against the rotatable barrel such that the rotatable barrel is forced upward away from the internal mount.

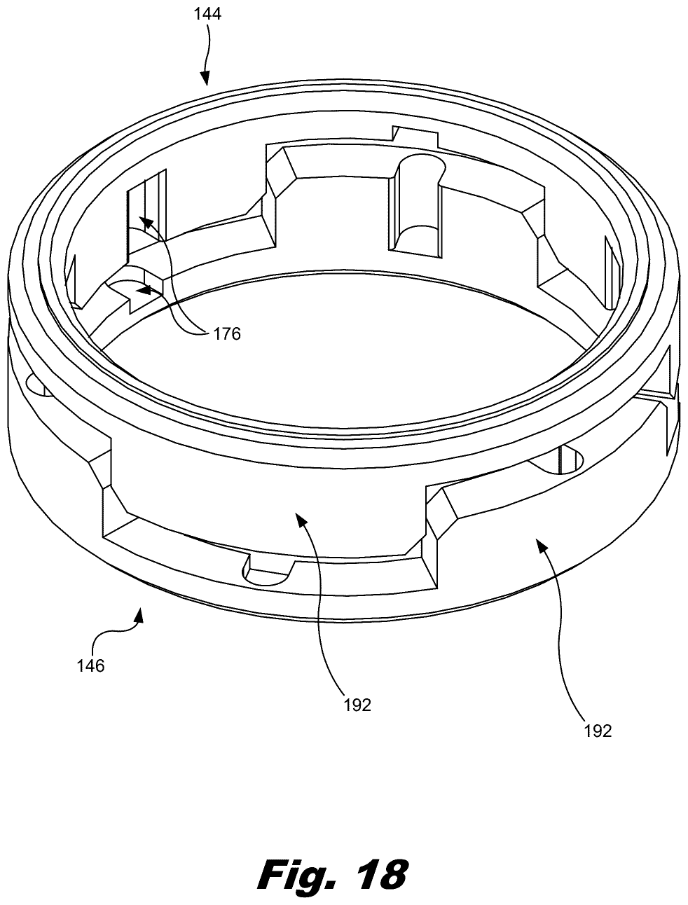

FIGS. 16 and 17 further shows that the lower spring cage 146 includes a plurality of spring cage interlocking teeth 192. In an embodiment, the lower spring cage 146 includes three spring cage interlocking teeth 192. As described herein, the spring cage interlocking teeth 192 of the lower spring cage 146 may interlock with similar spring cage interlocking teeth of an upper spring cage as shown in FIG. 18. FIG. 18 is a front perspective view of a lower spring cage 146 and an upper spring cage 144 of the prosthetic coupling device according to an embodiment of the present disclosure.

FIG. 18 shows the interaction between the upper spring cage 144 and the lower spring cage 146. In an embodiment, the shape of the upper spring cage 144 may match the lower spring cage 146 and may also include three spring cage interlocking teeth 192 that interlock with the three-spring cage interlocking teeth 192 of the lower spring cage 146. This interlocking of the spring cage interlocking teeth 192 between the lower spring cage 146 and the upper spring cage 144 may align the spring cage spring recesses 176 formed in the upper spring cage 144 and lower spring cage 146. The alignment of the spring cage spring recesses 176 allows for the spring cage springs to be placed within the spring cage spring recesses 176 so that the spring cage springs can force the lower spring cage 146 and upper spring cage 144 apart as described herein.

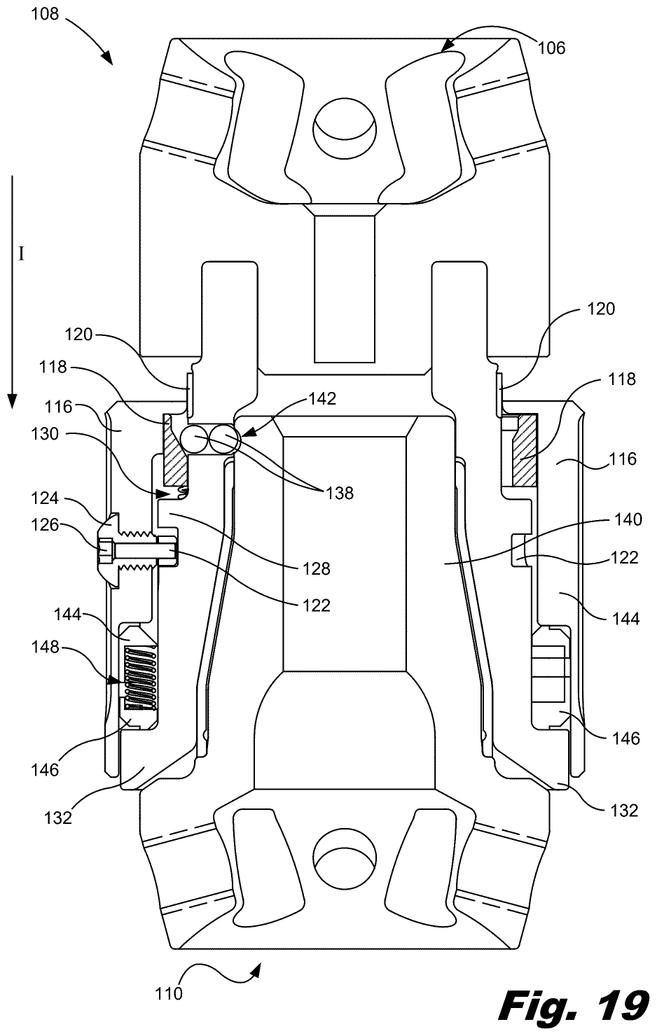

FIG. 19 is a side, cross-section view of a prosthetic coupling device 108 in an unlocked state according to another embodiment of the present disclosure. FIG. 19 is a cross-sectional plane along section line “B” of the prosthetic coupling device 108 similar to that shown in FIG. 3 but with the prosthetic coupling device 108 in the unlocked state as described herein. The elements of the prosthetic coupling device 108 are similar to those described in connection with FIG. 4 for example.

The prosthetic coupling device 108 includes an upper socket coupler 106 and lower socket coupler 110 used to couple an upper pylon/proximate shaft portion (not shown) and a lower pylon/distal shaft portion (not shown), respectively, to the prosthetic coupling device 108. Additionally, the prosthetic coupling device 108 includes the locking ring 118 formed around the internal mount 132 and within the rotatable barrel 116. In an embodiment, the locking ring 118 is operatively coupled to the rotatable barrel 116 such that rotation of the rotatable barrel 116 causes the locking ring 118 to also rotate the same degree. Further, the locking ring 118 interfaces with the locking ring spring 130 at the bottom surface of the locking ring 118. The locking ring spring 130 may be biased to force the locking ring 118 upwards and towards an interior surface of the rotatable barrel 116.

The prosthetic coupling device 108 further includes the upper spring cage 144 and the lower spring cage 146 described herein with one or more spring cage springs 148 formed between the upper spring cage 144 and lower spring cage 146. In an embodiment, the upper spring cage 144 and lower spring cage 146 may be mechanically affixed to an outer surface of the internal mount 132 such that the upper spring cage 144 and lower spring cage 146 do not rotate with the rotation of the rotatable barrel 116, for example.

The prosthetic coupling device 108 also includes the set screw 124 with its bolt 126 being passed through the rotatable barrel 116 to engage with, in the embodiment shown in FIG. 19, the second vertical slot formed at the second end of the horizontal portion of the bolt slot 122. During operation, the patient may press down on the rotatable barrel 116 in the direction of line “I” to pass the bolt 126 and set screw 124 downward along the vertical slot and into the horizontal portion of the bolt slot 122. By pressing down on the rotatable barrel 116, the patient overcomes the spring bias from the one or more locking ring springs 130 and one or more spring cage springs 148. As the patient rotates the rotatable barrel 116 in a direction (e.g., in a counterclockwise direction), the bolt 126 and a bolt bearing 128 formed on the end of the bolt 126 are passed along the entire length of the horizontal portion of the bolt slot 122 until this assembly reaches the second end of the horizontal portion of the bolt slot 122 and to a second vertical slot formed at a second end of the horizontal portion of the bolt slot 122 as shown in FIG. 19. In an embodiment, this second vertical slot formed at the second end of the horizontal portion of the bolt slot 122 may have a vertically lower terminal end than the first vertical slot preventing the rotatable barrel 116 from returning to a maximum height as shown in FIG. 4, for example. When the set screw 124/bolt 126 assembly is placed within the second vertical slot of the bolt slot 122, the prosthetic coupling device 108 is in an unlocked state.

In an embodiment, the prosthetic coupling device 108 may also include an indicator ring 120. In the embodiment shown in FIG. 19, the indicator ring 120 may be used by the patient to determine that the prosthetic coupling device 108 is in an unlocked state. As described herein, when the prosthetic coupling device 108 is in a locked state as shown in FIG. 4, the rotatable barrel 116 is in a maximum vertical position. With the rotatable barrel 116 in this maximum vertical position, the indicator ring 120 is not visible to the patient because the rotatable barrel 116 is physically covering the indicator ring 120. However, as described herein and shown in FIG. 19, when the prosthetic coupling device 108 is in an unlocked state, the position of the rotatable barrel 116 is altered such that the rotatable barrel 116 is at a lower position than when the prosthetic coupling device 108 is in a locked state. With the rotatable barrel 116 at this lower position, the indicator ring 120 is made visible by the patient. Therefore, in an embodiment, where the patient can see the indicator ring 120, the patient will understand that the prosthetic coupling device 108 is in an unlocked state and that the detachable coupler 140/lower pylon/distal shaft portion/prosthetic foot assembly may be removed from the remaining portions of the prosthetic coupling device 108. Even further, where the patient sees the indicator ring 120, the patient may understand that the patient should not place weight on the prosthetic device (e.g., FIG. 1, 100) or otherwise walk using the prosthetic device due to the detachable coupler 140 not being operatively locked to the remaining portions of the prosthetic coupling device 108 as described herein.

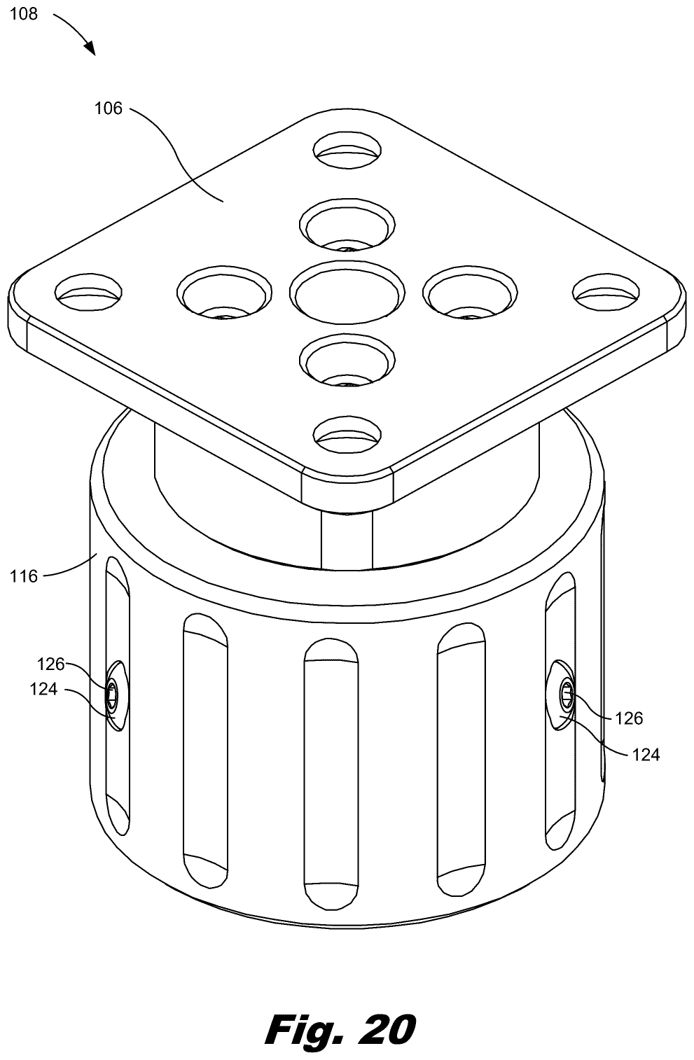

FIG. 20 is a perspective view of a prosthetic coupling device 108 according to another embodiment of the present disclosure. The prosthetic coupling device 108, show in FIG. 20 includes a socket coupler 106 operatively coupled to the internal mount 132 of the prosthetic coupling device 108. The socket coupler 158 may include a surface onto which other components of the prosthetic device (e.g., FIG. 1, 100) may be coupled to. In an embodiment, the socket coupler 106 is operatively coupled directly to a socket (e.g., FIG. 1, 102) of the prosthetic device. It is appreciated, however, that the socket coupler 106 may be used to be operatively coupled to other components of a prosthetic device. For example, the socket coupler 106 may be operatively coupled to a fixture directly implanted into a bone of a patient such as a femur bone. In this example embodiment, the socket coupler 106 may be operatively coupled to an abutment screw or other coupling device formed at a terminal end the fixture implanted into the patient's bone.

Similar to FIG. 4, FIG. 20 shows the rotatable barrel 116 rotatable in a counterclockwise direction, in an example embodiment, which causes the detachable coupler (not shown) to be removed from the prosthetic coupling device 108. Additionally, as described herein, the set screw 124 with its coaxial bolt 126 are also shown in FIG. 20 and are used to limit the rotational degree of the rotatable barrel and set a location where the ball bearing recesses align with the horizontal ball bearing shafts as described herein.

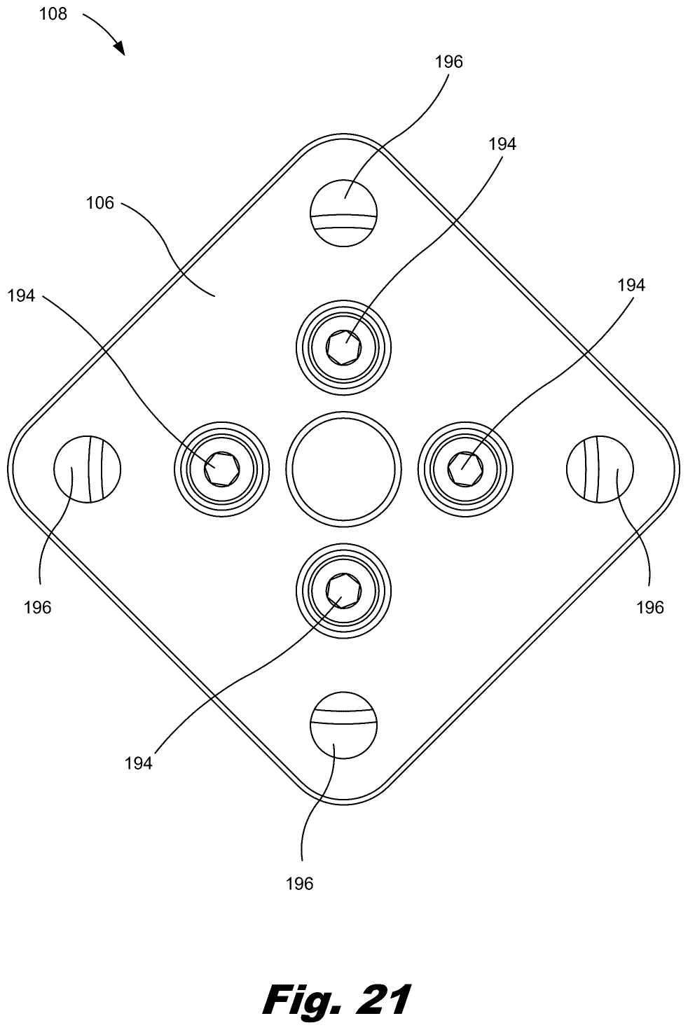

Further details of the socket coupler 106 are shown in FIG. 21. FIG. 21 is a top view of the prosthetic coupling device 108 that includes a socket coupler 106 according to another embodiment of the present disclosure. This top view of the prosthetic coupling device 108 and the socket coupler 106 shows that the socket coupler 106 is operatively coupled to the internal mount (not shown) via as set of socket coupler bolts 194. In the example embodiment shown in FIG. 21, four socket coupler bolts 194 is used to secure the socket coupler 106 to the internal mount and the remaining portions of the prosthetic coupling device 108. The socket coupler bolts 194 may include any diameter of bolts (e.g., either major diameter or minor diameter) that may be passed into socket coupler bolt vias (not shown) formed into a lip of the internal mount.

In the embodiment shown in FIG. 21, the socket coupler 106 includes one or more socket coupler coupling holes 196 used to operatively couple the socket coupler 106 and the remaining portions of the prosthetic coupling device 108 to a socket or other device of the prosthetic device. In an embodiment, the socket coupler coupling holes 196 may be threaded to receive a bolt or screw used to couple the socket coupler 106 to these other portions of the prosthetic device described herein. Additionally, or alternatively, the socket coupler holes 196 may be positioned such that a nut associated with those bolts or other fasteners may be used to secure the socket coupler 106 to the other components or devices of the prosthetic device as described herein. For example, the socket coupler bolts 194 may be used to secure the socket coupler 106 to a socket (e.g., FIG. 1, 102) or other components such as a vacuum pump used to create a vacuum between a liner around a patient's leg and a socket wall. In an embodiment, the socket coupler holes 196 may receive countersunk screws that, when fully seated into the socket coupler 106 to couple the socket coupler 106 to another component of the prosthetic device, is flat along a surface of the socket coupler 106. It is appreciated that other coupling devices apart from or an in addition to the socket coupler holes 196 may be used to couple the socket coupler 106 to a socket or other component of the prosthetic device and the present specification contemplates those other coupling devices.

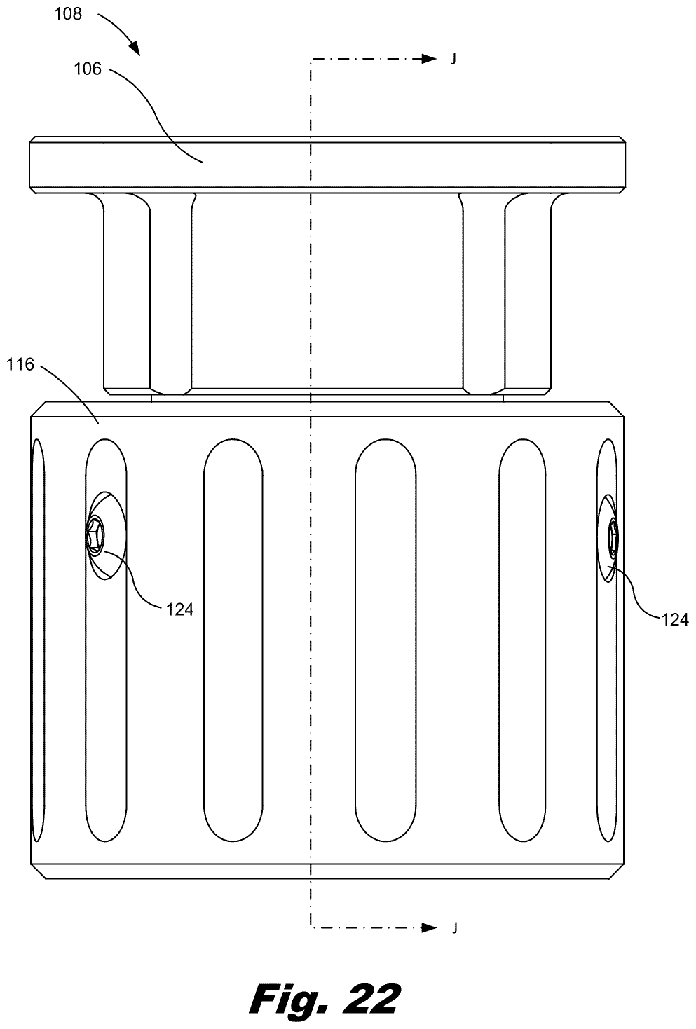

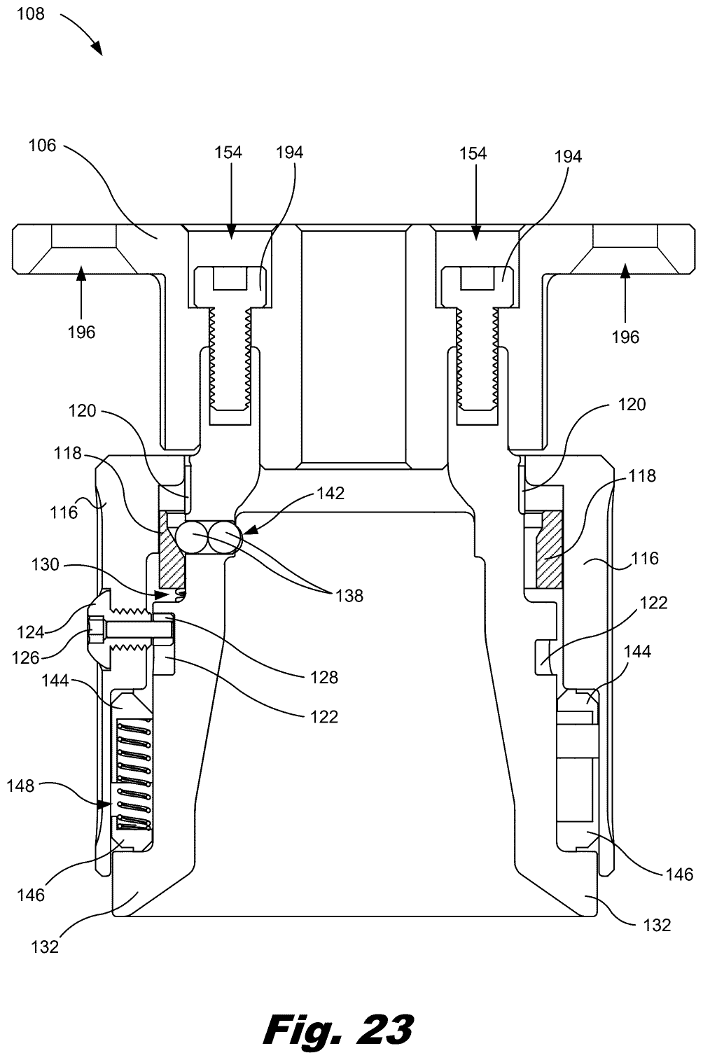

FIGS. 22 and 23 show further details of the socket coupler 106, the socket coupler bolts 194, and the socket coupler bolt vias 196 described herein. FIG. 22 is a side view of a prosthetic coupling device 108 showing the socket coupler 106 operatively coupled to the internal mount (not shown) of the prosthetic coupling device 108 according to another embodiment of the present disclosure. Additionally, FIG. 23 is a side, cross-section view of a prosthetic coupling device according to another embodiment of the present disclosure. FIG. 22 also includes a section line “J” indicating a cross-sectional plane of the prosthetic coupling device 108 with the socket coupler 106.

As shown in FIG. 22, the socket coupler 106 may be generally flat to interface with a relatively flat surface of a component of the prosthetic device to which the prosthetic coupling device 108 is to be coupled to. It is appreciated, however, that the surface of the socket coupler 106 may fit with other portions of those potential components of the prosthetic device and may include surfaces that increase a coefficient of friction or provide additional engineering fits between the socket coupler 106 and these other components of the prosthetic device.

Turning to FIG. 23, the cross-section side view of the prosthetic coupling device 108 is along section line “J” is shown in FIG. 23. Along with other elements of the prosthetic coupling device 108, his cross-section view shows the socket coupler 106 operatively coupled to the internal mount 132 of the prosthetic coupling device 108. As described herein, the socket coupler 106 is operatively coupled to the internal mount 132 via one or more socket coupler bolts 194. The socket coupler bolts 194 may be passed through and into one or more socket coupler bolt vias 198 formed into a lip portion of the internal mount 132. In an embodiment, the socket coupler bolt vias 198 may be threaded to receive threads from each of the socket coupler bolts 194. A thickness of the internal mount may exceed the diameter of the socket coupler bolts 194 (e.g., either major diameter or minor diameter)) such that the socket coupler bolts 194 can be seated into the internal mount 132 without compromising the structural integrity of the internal mount 132 as weight is placed on these components.

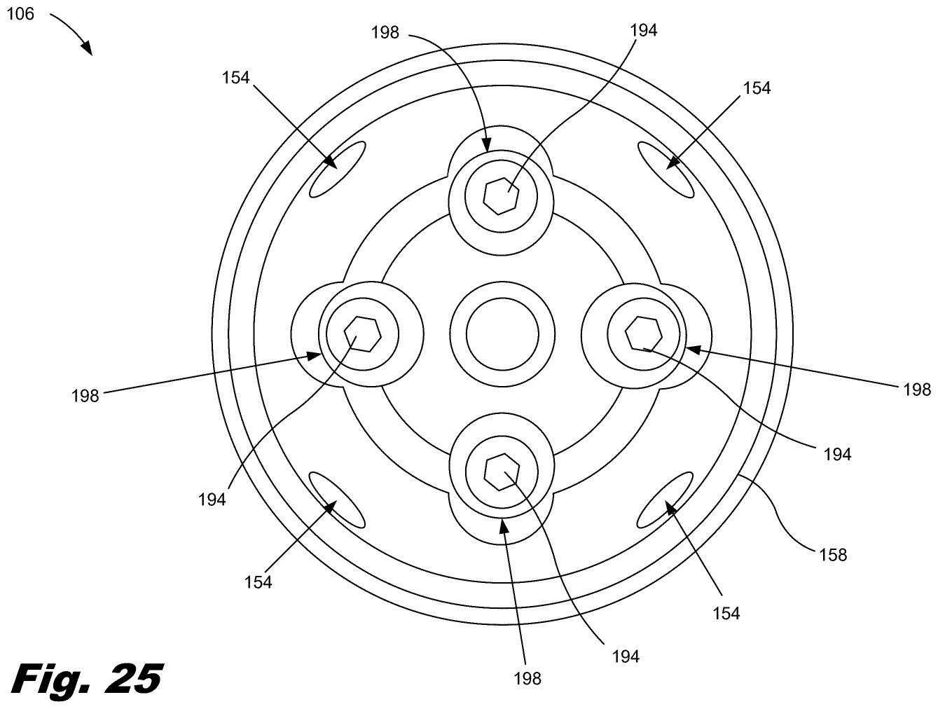

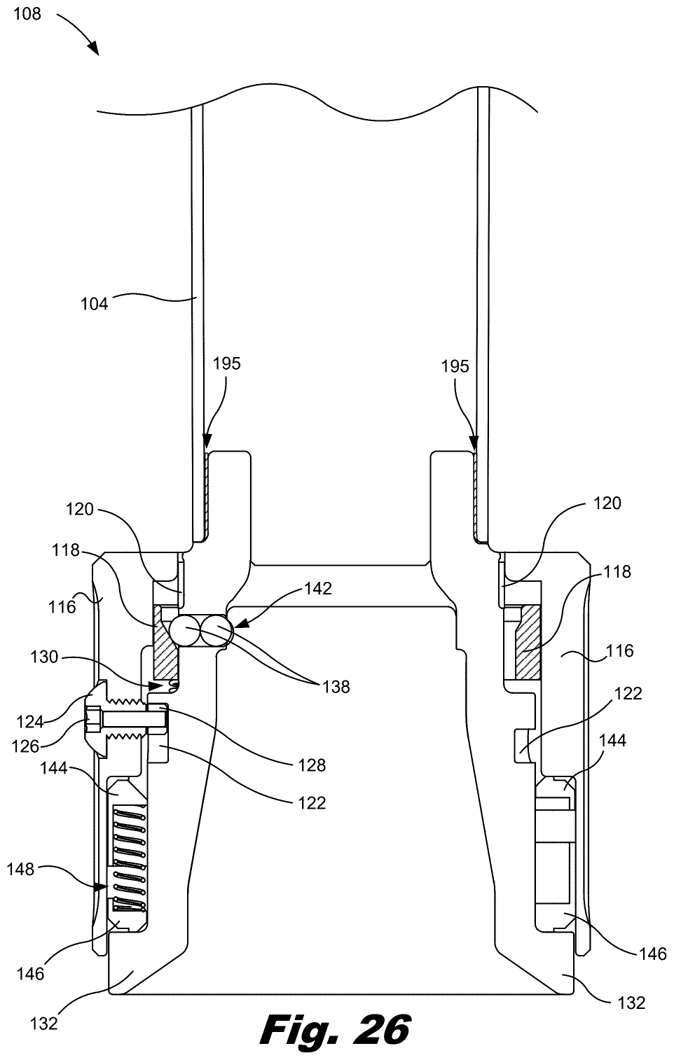

FIGS. 24 and 25 show another example embodiment of a socket coupler 106 that allows the prosthetic coupling device 108 to be interfaced with other components of the prosthetic device as described herein. FIG. 24 is a perspective view of a prosthetic coupling device 108 according to another embodiment of the present disclosure. Further, FIG. 25 is a top view of a prosthetic coupling device 108 according to another embodiment of the present disclosure. The prosthetic coupling device 108 shown in FIGS. 25 and 26 include a socket coupler 106. The socket coupler 106 shown in FIG. 24 may be a female pyramid coupler used to couple the prosthetic coupling device 108 to a pyramid-style terminator on a prosthetic device (e.g., FIG. 1, 100). In an embodiment, the socket coupler 106 also includes one or more socket coupler bolts 194 put into one or more socket coupler bolt vias 198 in order to secure the socket coupler 106 to the internal mount 132 of the prosthetic coupling device 108. In an embodiment, offset counterbores 197 may be formed into the prosthetic coupling device 108 to create the socket coupler bolt vias 198 on the socket coupler 106 so that a facilitating relief may be created for angle adjustment on the pyramid-style terminator on a prosthetic device (e.g., FIG. 1, 100).

The socket coupler 106 of FIG. 20 includes one or more socket coupler coupling holes 154 used to secure the pyramid-style terminator on a prosthetic device (e.g., FIG. 1, 100), for example, to the socket coupler 106 when the prosthetic coupling device 108 is coupled to the prosthetic device. In the example embodiment shown in FIG. 24, the socket coupler coupling holes 154 may be used to pass a bolt or other securing device through the socket coupler 106, into a central location of the socket coupler 106, and against the pyramid-style terminator on a prosthetic device or other coupling device that the socket coupler 106 may be coupled to.

Turning to FIG. 25, the socket coupler 106 includes, in an embodiment, four socket coupler bolts 194 each placed within a socket coupler bolt via 198. In an embodiment, the socket coupler bolt vias 198 may pass through the socket coupler 106 and into a lip of the internal mount (not shown). It is appreciated that more or less than four socket coupler bolts 194 and socket coupler bolt vias 198 may be used to couple the socket coupler 106 to the internal mount and the present specification contemplates the use of more or less than the four socket coupler bolts 194 shown in FIG. 25. The socket coupler 158 further includes four socket coupler coupling holes 166 used to secure the socket coupler 158 to terminal ends of the prosthetic device (e.g., FIG. 1, 100). Additionally, the present specification contemplates the use of more or less than the four socket coupler coupling holes 166 shown in FIG. 19 in order to secure a terminal end coupler on a prosthetic device to the socket coupler 158.

FIG. 26 is a side, cross-section view of a prosthetic coupling device 108 according to another embodiment of the present disclosure. The cross-section view of the prosthetic coupling device 108 may be similar to the cross-section view shown in FIG. 4 except, in FIG. 26, the internal mount 132 is coupled to an upper pylon 104 or other component of the prosthetic device. In FIG. 26, the internal mount 132 may have an upper portion that has a smaller outer diameter than an internal diameter of the upper pylon 104. This allows the upper pylon 104 to be placed around the upper portion of the internal mount 132 for the upper pylon 104 to be secured to the internal mount 132 of the prosthetic coupling device 108. In an embodiment, the upper pylon 104 may be pressed fit onto the upper portion of the internal mount 132 with the interface between the upper pylon 104 and the upper portion of the internal mount 132 being a press fit, a driving fit, or a forced fit among other types of engineering fits.

In the embodiment shown in FIG. 26, the upper pylon 104 may be operatively coupled to an upper portion of the internal mount 132 via application of a glue 195. This glue 195 may be applied to those surfaces where the interior surface of the upper pylon 104 interfaces with the exterior surface of the upper portion of the internal mount 132. As described herein, an industrial glue may be used to secure the upper pylon 104 to the upper portion of the internal mount 132. An example of an industrial glue that can be used to couple one or more components of the prosthetic coupling device 108 together may include 3M® Scotch-Weld® epoxy adhesive DP420 or DP 420NS or similar types of glues, epoxy, or chemical adhesives. Scotch-Weld® is a registered trademark of the 3M corporation of Maplewood, Minnesota. This glue 195 may bond the upper pylon 104 to the upper portion of the internal mount 132 to an extent to allow the patient to place weight onto the interface between the upper pylon 104 and the internal mount 132. In an embodiment where the prosthetic device is a leg prosthetic as shown in FIG. 1, for example, the entire patient's weight may be placed on the interface between the upper pylon 104 and the internal mount 132 as shown in FIG. 26 without the upper pylon 104 breaking away from the internal mount 132.

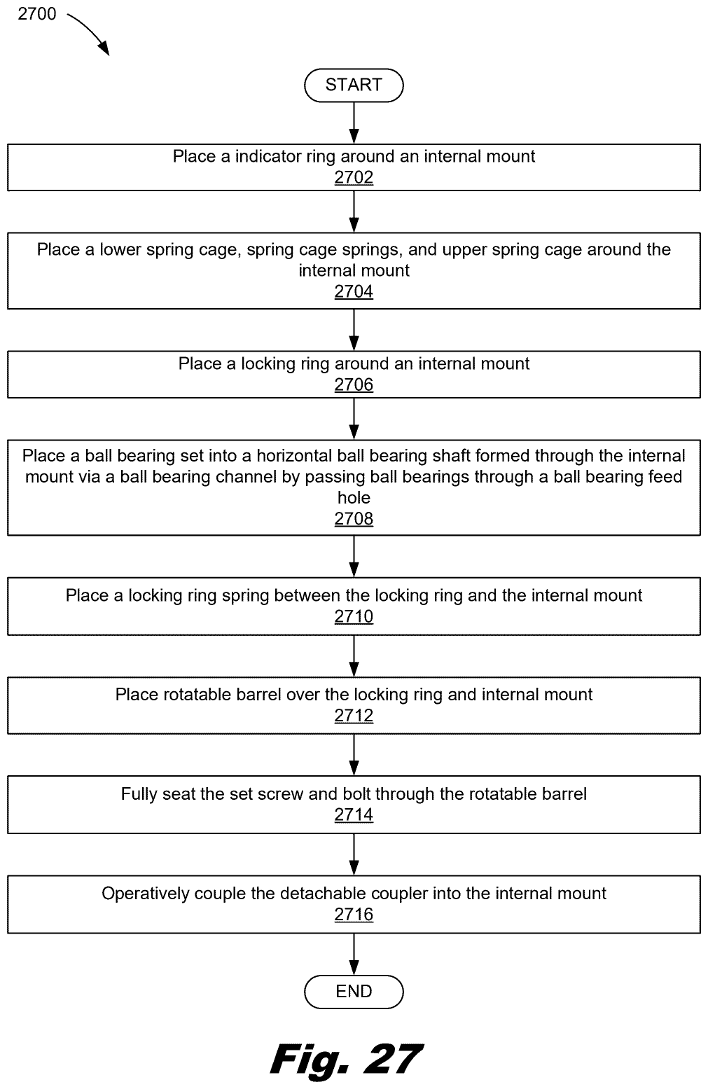

FIG. 27 is a flow diagram of a method 2700 of manufacturing a prosthetic device according to an embodiment of the present disclosure. The method 2700 described herein may include, at block 2702, placing an indicator ring onto an outer surface of an internal mount of the prosthetic coupling device. As described herein, when the prosthetic coupling device is in a locked state the indicator ring is not visible to the patient because the rotatable barrel is physically covering the indicator ring. However, when the prosthetic coupling device is in an unlocked state, the position of the rotatable barrel is altered such that the rotatable barrel is at a lower position with the indicator ring being viewable to the patient. Therefore, in an embodiment, where the patient can see the indicator ring, the patient will understand that the prosthetic coupling device is in an unlocked state. In an embodiment, the indicator ring may be glued to the external surface of the internal mount. In another embodiment, the indicator ring may be force fit onto the external surface of the internal mount using an engineering fit that prevents the removal of the indicator ring from the internal mount. In another embodiment, the indicator ring 120 may be operatively coupled to the external surface of the internal mount 132 using any type of fastening device such as a nail, screw, or bolt among others.

At block 2704, the method 2700 further includes placing the lower spring cage, the spring cage springs, and upper spring cage around the internal mount. In an embodiment, the upper spring cage and lower spring cage may be mechanically affixed to an outer surface of the internal mount such that the upper spring cage and lower spring cage do not rotate with the rotation of the rotatable barrel, for example. In these embodiments, the upper spring cage and lower spring cage may be operatively coupled to the external surface of the internal mount using a coupling device (bolt, screw, nail, etc.), a glue, or via an engineering fit that prevents movement of the upper spring cage and lower spring cage relative to the internal mount.

At block 2706, a locking ring is placed around the internal mount of the prosthetic coupling device. As described herein, the locking ring may include a ball bearing feed hole. At block 2708, the method 2700 includes placing at least one ball bearing set into a horizontal ball bearing shaft formed through the internal mount via the ball bearing channel by passing ball bearings through the ball bearing feed hole. The ball bearing feed hole may be used to introduce the ball bearings into their respective horizontal ball bearing shafts during manufacturing and assembly of the prosthetic coupling device. During assembly, a manufacturer may pass a sufficient number of ball bearings through the ball bearing feed hole such that the space within the horizontal ball bearing shafts is filled with ball bearings. Once a sufficient number of ball bearings have been introduced through the ball bearing feed hole and into their respective horizontal ball bearing shafts, the ball bearing feed hole may be capped off and the rotatable barrel may be slipped over the locking ring thereby preventing the ball bearings from leaving their respective horizontal ball bearing shafts.

The method 2700 may further include placing a locking ring spring between the locking ring and the internal mount at block 2710. As described herein, this locking ring spring may be biased to force the locking ring upwards. At block 2712, the rotatable barrel may be placed over the locking ring and internal mount. In an embodiment, the locking ring includes a plurality of barrel interlocking surfaces. The barrel interlocking surfaces are formed on the exterior surface of the locking ring. As described herein, the barrel interlocking surfaces interface with complementary locking surfaces formed on the internal surface of the rotatable barrel. The mechanical engagement between the barrel interlocking surfaces and those complementary locking surfaces formed on the internal surface of the rotatable barrel causes the locking ring to rotate with the rotatable barrel during operation by the patient.

At block 2714, the set screw and bolt may be fully seated through the rotatable barrel. In an embodiment, the set screw with its bolt 126 are passed through the rotatable barrel and engages with the bolt slot formed on an exterior surface of the internal mount. In an embodiment, the bolt may be fitted with a bolt bearing. The bolt bearing may be allowed to rotate about the axis of the bolt such that as the rotatable barrel is rotated, the bolt bearing engages with the surfaces of the bolt slot to allow for the bolt and bolt bearing to pass along the surface of the bolt slot to reduce friction between the bolt and the surfaces of the bolt slot. Additionally, the diameter of the bolt bearing may be selected such that as the bolt and bolt bearing reach the vertical portions of the bolt slot, the bolt bearing fits and is seated within the vertical portions of the bolt slot.

The method 2700 also includes operatively coupling the detachable coupler in the internal mount at block 2716. This process may be similar to what the patient engages in as the patient couples the lower pylon, foot, and detachable coupler to the remaining portions of the prosthetic coupling device including the internal mount and rotatable barrel as well as those other components described herein. As the detachable coupler is inserted into the internal mount, the ball bearing set may be moved radially away from the detachable coupler because the rotatable barrel has been placed in an unlocked position by the rotation of the rotatable barrel in a counterclockwise direction in the embodiments described herein. Once the detachable coupler is oriented properly via interaction of the registering post on the detachable coupler and the registering slot of the internal mount, the coupler detents are aligned with the horizontal ball bearing shafts that house the ball bearing sets.

The manufacturer or assembler of the prosthetic coupling device may then rotate the rotatable barrel in a clockwise direction to place the rotatable barrel in a locked position. Again, these mechanical functions resulting from the rotation of the rotatable barrel either clockwise or counterclockwise by the patient allows the patient to disconnect the lower pylon, foot, and detachable coupler assembly from the remaining portions of the prosthetic device. In an example embodiment, the patient may own a first lower pylon and prosthetic foot that has a first type of shoe on the prosthetic foot such as a tennis shoe or sneaker. In this example embodiment, the patient may also own a second lower pylon and prosthetic foot combination that includes a dress shoe on the prosthetic foot. With the prosthetic coupling device, the patient may easily switch from the first lower pylon and prosthetic foot/shoe combination with the second lower pylon and prosthetic foot/shoe by rotating the rotatable barrel of the prosthetic coupling device in a first direction, removing the first lower pylon and prosthetic foot/shoe combination from the prosthetic coupling device, inserting the second lower pylon and prosthetic foot/shoe combination into the prosthetic coupling device, and turning the rotatable barrel in a second direction to lock the second lower pylon and prosthetic foot/shoe combination to the prosthetic coupling device and the upper portions of the prosthetic device. It is also clear that other, differently fitted, lower pylon and prosthetic foot combinations may be owned by the patient to quickly switch out different footwear when needed. Additionally, the prosthetic coupling device allows the patient to know when the lower pylon/prosthetic foot and securely locked to the prosthetic coupling device when a maximum rotation (e.g., a quarter turn rotation) of the rotatable barrel is completed through the use of the indicator ring. At this point, the method 2700 may end.

The blocks of the flow diagrams of FIG. 27 or steps and aspects of the operation of the embodiments herein and discussed above need not be performed in any given or specified order. It is contemplated that additional blocks, steps, or functions may be added, some blocks, steps or functions may not be performed, blocks, steps, or functions may occur contemporaneously, and blocks, steps, or functions from one flow diagram may be performed within another flow diagram.

Although only a few exemplary embodiments have been described in detail herein, those skilled in the art will readily appreciate that many modifications are possible in the exemplary embodiments without materially departing from the novel teachings and advantages of the embodiments of the present disclosure. Accordingly, all such modifications are intended to be included within the scope of the embodiments of the present disclosure as defined in the following claims. In the claims, means-plus-function clauses are intended to cover the structures described herein as performing the recited function and not only structural equivalents, but also equivalent structures.

The above-disclosed subject matter is to be considered illustrative, and not restrictive, and the appended claims are intended to cover any and all such modifications, enhancements, and other embodiments that fall within the scope of the present invention. Thus, to the maximum extent allowed by law, the scope of the present invention is to be determined by the broadest permissible interpretation of the following claims and their equivalents and shall not be restricted or limited by the foregoing detailed description.

1. A prosthetic coupling device comprising:
an internal mount;
a rotatable barrel formed around the internal mount;
a locking ring formed between the internal mount and the rotatable barrel and mechanically affixed to the rotatable barrel to be rotatably moveable with the rotatable barrel;
a horizontal ball bearing shaft formed through the internal mount, the horizontal ball bearing shaft housing a ball bearing set wherein the ball bearing set interfaces with the locking ring such that the ball bearing set moves radially inward or outward based on the radial orientation of the locking ring and rotatable barrel; and
a detachable coupler formed to fit within the mounting portion of the internal mount, the detachable coupler including a coupler detent formed on a radially external surface of the detachable coupler, wherein the ball bearing set interfaces with the coupler detent to lock the detachable coupler to the internal mount when the locking ring is radially oriented to move the ball bearing set radially inward.

2. The prosthetic coupling device of claim 1 further comprising:
an upper spring cage;
a lower spring cage; and
a spring cage spring placed between the upper spring cage and the lower spring cage to create a biasing force against the rotatable barrel upwards to interface the locking ring with the ball bearing set to move the ball bearing set radially inward.

3. The prosthetic coupling device of claim 1 further comprising:
a locking ring spring to apply an upward biasing force against a bottom surface of the locking ring from a surface of the internal mount.

4. The prosthetic coupling device of claim 1 further comprising:
a bolt slot formed on an exterior surface of the internal mount;
a set screw formed through the rotatable barrel, the set screw having:
a head and a shaft, the shaft comprising outer threads;
a bolt channel through the longitudinal axis of the shaft; and
internal threads formed on an internal surface of the bolt channel; and
a bolt placed coaxially within the bolt channel, the length of the bolt extending past a terminal end of the set screw to extend into the bolt slot.

5. The prosthetic coupling device of claim 4 further comprising:
the bolt slot including:
a horizontal portion;
first vertical slot formed at a first end of the horizontal portion of the bolt slot to receive an end of the bolt in a first radial orientation of the rotatable barrel; and
a second vertical slot formed at a second end of the horizontal portion of the bolt slot to receive the end of the bolt in a second radial orientation of the rotatable barrel.

6. The prosthetic coupling device of claim 4 further comprising:
a bolt bearing formed on an end of the bolt to slide the bolt along the bolt channel.

7. The prosthetic coupling device of claim 1 further comprising:
a registering slot formed into the internal mount to receive a registering pin formed on the detachable coupler to align the detachable coupler radially relative to the internal mount.

8. The prosthetic coupling device of claim 1 further comprising:
an indicator ring formed onto an exterior surface of the internal mount wherein the indicator ring is:
viewable to the patient when the ball bearing set is not interfacing with the coupler detents and the detachable coupler is not in a locked position with the internal mount; and
not viewable to the patient when the ball bearing set is interfacing with the coupler detents and the detachable coupler is in a locked position with the internal mount.

9. The prosthetic coupling device of claim 1 further comprising:
the detachable coupler including a lower socket coupler to couple a lower pylon to the detachable coupler; and
the internal mount including an upper socket coupler to couple an upper pylon to the internal mount.

10. A coupling device comprising:
an internal mount;
a rotatable barrel formed around the internal mount;
a locking ring formed between the internal mount and the rotatable barrel and mechanically affixed to the rotatable barrel to be rotatably moveable with the rotatable barrel;
a horizontal ball bearing shaft formed through the internal mount, the horizontal ball bearing shaft housing a ball bearing set wherein the ball bearing set interfaces with the locking ring such that the ball bearing set moves radially inward or outward based on the radial orientation of the locking ring and rotatable barrel;
a detachable coupler formed to fit within the mounting portion of the internal mount, the detachable coupler including a coupler detent formed on a radially external surface of the detachable coupler, wherein the ball bearing set interfaces with the coupler detent to lock the detachable coupler to the internal mount when the locking ring is radially oriented to move the ball bearing set radially inward; and
an upper spring cage, a lower spring cage, and a spring cage spring placed between the upper spring cage and the lower spring cage to create a biasing force against the rotatable barrel upwards to interface the locking ring with the ball bearing set to move the ball bearing set radially inward.

11. The coupling device of claim 10 further comprising:
a locking ring spring to apply an upward biasing force against a bottom surface of the locking ring from a surface of the internal mount.

12. The coupling device of claim 10 further comprising:
a bolt slot formed on an exterior surface of the internal mount;
a set screw formed through the rotatable barrel, the set screw having:
a head and a shaft, the shaft comprising outer threads;
a bolt channel through the longitudinal axis of the shaft; and
internal threads formed on an internal surface of the bolt channel; and
a bolt placed coaxially within the bolt channel, the length of the bolt extending past a terminal end of the set screw to extend into the bolt slot.

13. The coupling device of claim 12 further comprising:
the bolt slot including:
a horizontal portion;
a first vertical slot formed at a first end of the horizontal portion of the bolt slot to receive an end of the bolt in a first radial orientation of the rotatable barrel; and
a second vertical slot formed at a second end of the horizontal portion of the bolt slot to receive the end of the bolt in a second radial orientation of the rotatable barrel.

14. The coupling device of claim 12 further comprising:
a bolt bearing formed on an end of the bolt to slide the bolt along the bolt channel.

15. The coupling device of claim 10 further comprising:
a registering slot formed into the internal mount to receive a registering pin formed on the detachable coupler to align the detachable coupler radially relative to the internal mount.

16. The coupling device of claim 10 further comprising:
an indicator ring formed onto an exterior surface of the internal mount wherein the indicator ring is:
viewable to the patient when the ball bearing set is not interfacing with the coupler detents and the detachable coupler is not in a locked position with the internal mount; and
not viewable to the patient when the ball bearing set is interfacing with the coupler detents and the detachable coupler is in a locked position with the internal mount.

17. A prosthetic coupling device comprising:
an internal mount;
a rotatable barrel formed around the internal mount;
a locking ring formed between the internal mount and the rotatable barrel and mechanically affixed to the rotatable barrel to be rotatably moveable with the rotatable barrel;
a locking ring spring to apply an upward biasing force against a bottom surface of the locking ring from a surface of the internal mount;
a horizontal ball bearing shaft formed through the internal mount, the horizontal ball bearing shaft housing a ball bearing set wherein the ball bearing set interfaces with the locking ring such that the ball bearing set moves radially inward or outward based on the radial orientation of the locking ring and rotatable barrel;
a detachable coupler formed to fit within the mounting portion of the internal mount, the detachable coupler including a coupler detent formed on a radially external surface of the detachable coupler, wherein the ball bearing set interfaces with the coupler detent to lock the detachable coupler to the internal mount when the locking ring is radially oriented to move the ball bearing set radially inward; and
an upper spring cage, a lower spring cage, and a spring cage spring placed between the upper spring cage and the lower spring cage to create a biasing force against the rotatable barrel upwards to interface the locking ring with the ball bearing set to move the ball bearing set radially inward.

18. The prosthetic coupling device of claim 17 further comprising:
a bolt slot formed on an exterior surface of the internal mount;
a set screw formed through the rotatable barrel, the set screw having:
a head and a shaft, the shaft comprising outer threads;
a bolt channel through the longitudinal axis of the shaft; and
internal threads formed on an internal surface of the bolt channel; and
a bolt placed coaxially within the bolt channel, the length of the bolt extending past a terminal end of the set screw to extend into the bolt slot.

19. The prosthetic coupling device of claim 18 further comprising:
the bolt slot including:
a horizontal portion;
a first vertical slot formed at a first end of the horizontal portion of the bolt slot to receive an end of the bolt in a first radial orientation of the rotatable barrel; and
a second vertical slot formed at a second end of the horizontal portion of the bolt slot to receive the end of the bolt in a second radial orientation of the rotatable barrel.

20. The prosthetic coupling device of claim 17 further comprising:
a bolt bearing formed on an end of the bolt to slide the bolt along the bolt channel.
